SEQUENCE LISTING (19 sequences)

SEQ ID NO: 1 - RNA, 8069 nt, Artificial Sequence
  GBV-B Replicon
accacaaacacuccaguuuguuacacuccgcuaggaaugcuccuggagcacccccccuagcagggcgugggggauuuccccugcccgucugcagaaggguggagccaaccaccuuaguauguaggcggcgggacucaugacgcucgcgugaugacaagcgccaagcuugacuuggauggcccugaugggcguucauggguucgguggugguggcgcuuuaggcagccuccacgcccaccaccucccagauagagcggcggcacuguagggaagaccggggaccggucacuaccaaggacgcagaccucuuuuugaguaucacgccuccggaaguaguugggcaagcccaccuauauguguugggaugguugggguuagccauccauaccguacugccugauaggguccuugcgaggggaucugggagucucguagaccguagcacaugccuguuauuucuacucaaacaaguccuguaccugcgcccagaacgcgcaagaacaagcagacggggcgcgccaugauugaacaagauggauugcacgcagguucuccggccgcuuggguggagaggcuauucggcuaugacugggcacaacagacaaucggcugcucugaugccgccguguuccggcugucagcgcaggggcgcccgguucuuuuugucaagaccgaccuguccggugcccugaaugaacugcaggacgaggcagcgcggcuaucguggcuggccacgacgggcguuccuugcgcagcugugcucgacguugucacugaagcgggaagggacuggcugcuauugggcgaagugccggggcaggaucuccugucaucucaccuugcuccugccgagaagguauccaucauggcugaugcaaugcggcggcugcauacgcuugauccggcuaccugcccauucgaccaccaagcgaaacaucgcaucgagcgagcacguacucggauggaagccggucuugucgaucaggaugaucuggacgaggagcaucaggggcucgcgccagccgaacuguucgccaggcucaaggcgcgcaugcccgacggcgaggaucucgucgugacccauggcgaugccugcuugccgaauaucaugguggaaaauggccgcuuuucuggauucaucgacuguggccggcuggguguggcggaccgcuaucaggacauagcguuggcuacccgugauauugcugaggagcuuggcggcgaaugggcugaccgcuuccucgugcuuuacgguaucgccgcucccgauucgcagcgcaucgccuucuaucgccuucuugacgaguucuucugaguuuaaacagaccacaacgguuucccucuagcgggaucaauuccgccccucucccuccccccccccuaacguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgauaauaccauggcaccuuuuacgcugcagugucucucugaacguggcacgcugucagcgauggcaguggucaugacugguauagacccccgaacuuggacuggaacuaucuucagauuaggaucucuggccacuagcuacaugggauuuguuugugacaacguguuguauacugcucaccauggcagcaaggggcgccgguuggcucaucccacaggcuccauacacccaauaaccguugacgcggcuaaugaccaggacaucuaucaaccaccauguggagcugggucccuuacucggugcucuugcggggagaccaagggguaucugguaacacgacuggggucauugguugaggucaauaaauccgaugacccuuauuggugugugugcggggcccuucccauggcuguugccaaggguucuucaggugccccgauucugugcuccuccgggcauguuauugggauguucaccgcugcuagaaauucuggcgguucagucagccagauuaggguuaggccguuggugugugcuggauaccauccccaguacacagcacaugccacucuugauacaaaaccuacugugccuaacgaguauucagugcaaauuuuaauugcccccacuggcagcggcaagucaaccaaauuaccacuuucuuacaugcaggagaaguaugaggucuugguccuaaaucccaguguggcuacaacagcaucaaugccaaaguacaugcacgcgacguacggcgugaauccaaauugcuauuuuaauggcaaauguaccaacacaggggcuucacuuacguacagcacauauggcauguaccugaccggagcauguucccggaacuaugauguaaucauuugugacgaaugccaugcuaccgaugcaaccaccguguugggcauuggaaagguccuaaccgaagcuccauccaaaaauguuaggcuagugguucuugccacggcuacccccccuggaguaaucccuacaccacaugccaacauaacugagauucaauuaaccgaugaaggcacuauccccuuucauggaaaaaagauuaaggaggaaaaucugaagaaagggagacaccuuaucuuugaggcuaccaaaaaacacugugaugagcuugcuaacgaguuagcucgaaagggaauaacagcugucucuuacuauaggggaugugacaucucaaaaaucccugagggcgacuguguaguaguugccacugaugccuuguguacaggguacacuggugacuuugauuccguguaugacugcagccucaugguagaaggcacaugccauguugaccuugacccuacuuucaccauggguguucgugugugcgggguuucagcaauaguuaaaggccagcguaggggccgcacaggccgugggagagcuggcauauacuacuauguagacgggaguuguaccccuucggguaugguuccugaaugcaacauuguugaagccuucgacgcagccaaggcaugguaugguuugucaucaacagaagcucaaacuauucuggacaccuaucgcacccaaccuggguuaccugcgauaggagcaaauuuggacgagugggcugaucucuuuucaauggucaaccccgaaccuucauuugucaauacugcaaaaagaacugcugacaauuauguuuuguugacugcagcccaacuacaacugugucaucaguauggcuaugcugcucccaaugacgcaccacgguggcagggagcccggcuugggaaaaaaccuugugggguucuguggcgcuuggacggcgcugacgccuguccuggcccagagcccagcgaggugaccagauaccaaaugugcuucacugaagucaauacuucugggacagccgcacucgcuguuggcguuggaguggcuauggcuuaucuagccauugacacuuuuggcgccacuugugugcggcguugcuggucuauuacaucagucccuaccggugcuacugucgccccagugguugacgaagaagaaaucguggaggagugugcaucauucauucccuuggaggccaugguugcugcaauugacaagcugaagaguacaaucaccacaacuaguccuuucacauuggaaaccgcccuugaaaaacuuaacaccuuucuugggccucaugcagcuacaauccuugcuaucauagaguauugcuguggcuuagucacuuuaccugacaaucccuuugcaucaugcguguuugcuuucauugcggguauuacuaccccacuaccucacaagaucaaaauguuccugucauuauuuggaggcgcaauugcguccaagcuuacagacgcuagaggcgcacuggcguucaugauggccggggcugcgggaacagcucuugguacauggacaucgguggguuuugucuuugacaugcuaggcggcuaugcugccgccucauccacugcuugcuugacauuuaaaugcuugaugggugaguggcccacuauggaucagcuugcugguuuagucuacuccgcguucaacccggccgcaggaguugugggcguuuugucagcuugugcaauguuugcuuugacaacagcagggccagaucacuggcccaacagacuucuuacuaugcuugcuaggagcaacacuguauguaaugaguacuuuauugccacucgugacauccgcaggaagauacugggcauucuggaggcaucuacccccuggagugucauaucagcuugcauccguuggcuccacaccccgacggaggaugauugcggccucauugcuuggggucuagagauuuggcaguacgugugcaauuucuuugugauuugcuuuaauguccuuaaagcuggaguucagagcaugguuaacauuccugguuguccuuucuacagcugccagaagggguacaagggccccuggauuggaucagguaugcuccaagcacgcuguccaugcggugcugaacucaucuuuucuguugagaaugguuuugcaaaacuuuacaaaggacccagaacuuguucaaauuacuggagaggggcuguuccagucaacgcuaggcugugugggucggcuagaccggacccaacugauuggacuagucuugucgucaauuauggcguuagggacuacuguaaauaugagaaauugggagaucacaucuuuguuacagcaguauccucuccaaaugucuguuucacccaggugcccccaaccuugagagcugcaguggccguggacggcguacagguucaguguuaucuaggugagcccaaaacuccuuggacgacaucugcuugcuguuacgguccugacgguaaggguaaaacuguuaagcuucccuuccgcguugacggucacacaccuggugugcgcaugcaacuuaauuugcgugaugcacuugagacaaaugacuguaauuccacaaacaacacuccuagugaugaagccgcaguguccgcucuuguuuucaaacaggaguugcggcguacaaaccaauugcuugaggcaauuucagcuggcguugacaccaccaaacugccagcccccuccaucgaagagguagugguaagaaagcgccaguuccgggcaagaacugguucgcuuaccuugccccccccuccgagauccgucccaggagugucauguccugaaagccugcaacgaagugacccguuagaagguccuucaaaccucccuccuucaccaccuguucuacaguuggccaugccgaugccccuguugggagcgggugaguguaacccuuucacugcaauuggaugugcaaugaccgaaacaggcggaggcccugaugauuuacccaguuacccucccaaaaaggaggucucugaauggucagacgaaaguuggucaacggcuacaaccgcuuccagcuacguuacuggccccccguacccuaagauacggggaaaggauuccacucagucagcccccgccaaacggccuacaaaaaagaaguugggaaagagugaguuuucgugcagcaugagcuacacuuggaccgacgugauuagcuucaaaacugcuucuaaaguucugucugcaacucgggccaucacuagugguuuccucaaacaaagaucauugguguaugugacugagccgcgggaugcggagcuuagaaaacaaaaagucacuauuaauagacaaccucuguuccccccaucauaccacaagcaagugagauuggcuaaggagaaagcuucaaaaguugucggugucaugugggacuaugaugaaguagcagcucacacgcccucuaagucugcuaagucccacaucacuggccuucggggcacugauguucguucuggagcagcccgcaaggcuguucuggacuugcagaagugugucgaggcaggugagauaccgagucauuaucggcaaacagugauaguuccaaaggaggaggucuucgugaagaccccccagaaaccaacaaagaaacccccaaggcucaucucguacccccaccuugaaaugagauguguugagaagauguacuacggucagguugcuccugacguaguuaaagcugucaugggagaugcguacggguuuguagauccacguacccgugucaagcgucuguugucgauguggucacccgaugcagucggagccacaugcgauacaguguguuuugacaguaccaucacacccgaggauaucaugguggagacagacaucuacucagcagcuaaacucagugaccaacaccgagcuggcauucacaccauugcgaggcaguuauacgcuggaggaccgaugaucgcuuaugauggccgagagaucggauaucguagguguaggucuuccggcgucuauacuaccucaaguuccaacaguuugaccugcuggcugaagguaaaugcugcagccgaacaggcuggcaugaagaacccucgcuuccuuauuugcggcgaugauugcaccguaauuuggaaaagcgccggagcagaugcagacaaacaagcaaugcgugucuuugcuagcuggaugaaggugaugggugcaccacaagauugugugccucaacccaaauacaguuuggaagaauuaacaucaugcucaucaaauguuaccucuggaauuaccaaaaguggcaagccuuacuacuuucuuacaagagauccucguaucccccuuggcaggugcucugccgagggucugggauacaaccccagugcugcguggauuggguaucuaauacaucacuacccauguuuguggguuagccguguguuggcuguccauuucauggagcagaugcucuuugaggacaaacuucccgagacugugaccuuugacugguaugggaaaaauuauacggugccuguagaagaucugcccagcaucauugcuggugugcacgguauugaggcuuucucgguggugcgcuacaccaacgcugagauccucagaguuucccaaucacuaacagacaugaccaugcccccccugcgagccuggcgaaagaaagccagggcgguccucgccagcgccaagaggcguggcggagcacacgcaaaauuggcucgcuuccuucucuggcaugcuacaucuagaccucuaccagauuuggauaagacgagcguggcucgguacaccacuuucaauuauugugauguuuacuccccggagggggauguguuuguuacaccacagagaagauugcagaaguuucuugugaaguauuuggcugucauuguuuuugcccuagggcucauugcuguuggauuagccaucagcugaacccccaaauucaaaauuaacuaacaguuuuuuuuuuuuuuuuuuuuuagggcagcggcaacaggggagaccccgggcuuaacgaccccgccgaugugaguuuggcgaccaugguggaucagaaccguuucgggugaagccauggucugaaggggaugacgucccuucuggcucauccacaaaaaccgucucgggugggugaggaguccuggcugugugggaagcagucaguauaauucccgucguguguggugacgccucacgacguacuuguccgcugugcagagcguaguaccaagggcugcaccccgguuuuuguuccaagcggagggcaacccccgcuuggaauuaaaaacu

SEQ ID NO: 2 - DNA, 9397 bp, Artificial Sequence
  GBV-B Replicon
accacaaacactccagtttgttacactccgctaggaatgctcctggagcaccccccctagcagggcgtgggggatttcccctgcccgtctgcagaagggtggagccaaccaccttagtatgtaggcggcgggactcatgacgctcgcgtgatgacaagcgccaagcttgacttggatggccctgatgggcgttcatgggttcggtggtggtggcgctttaggcagcctccacgcccaccacctcccagatagagcggcggcactgtagggaagaccggggaccggtcactaccaaggacgcagacctctttttgagtatcacgcctccggaagtagttgggcaagcccacctatatgtgttgggatggttggggttagccatccataccgtactgcctgatagggtccttgcgaggggatctgggagtctcgtagaccgtagcacatgcctgttatttctactcaaacaagtcctgtacctgcgcccagaacgcgcaagaacaagcagacgcaggcttcatatcctgtgtccattaaaacatctgttgaaaggggacaacgagcaaagcgcaaagtccagcgcgatgctcggcctcgtaattacaaaattgctggtatccatgatggcttgcagacattggctcaggctgctttgccagctcatggttggggacgccaagaccctcgccataagtctcgcaatcttggaatccttctggattaccctttggggtggattggtgatgttacaactcacacacctctagtaggcccgctggtggcaggagcggtcgttcgaccagtctgccagatagtacgcttgctggaggatggagtcaactgggctactggttggttcggtgtccacctttttgtggtatgtctgctatctttggcctgtccctgtagtggggcgcgggtcactgacccagacacaaataccacaatcctgaccaattgctgccagcgtaatcaggttatctattgttctccttccacttgtctacacgagcctggttgtgtgatctgtgcggacgagtgctgggttcccgccaatccgtacatctcacacccttccaattggactggcacggactccttcttggctgaccacattgattttgttatgggcgctcttgtgacctgtgacgcccttgacattggtgagttgtgtggtgcgtgtgtattagtcggtgactggcttgtcaggcactggcttattcacatagacctcaatgaaactggtacttgttacctggaagtgcccactggaatagatcctgggttcctagggtttatcgggtggatggccggcaaggtcgaggctgtcatcttcttgaccaaactggcttcacaagtaccatacgctattgcgactatgtttagcagtgtacactacctggcggttggcgctctgatctactatgcctctcggggcaagtggtatcagttgctcctagcgcttatgctttacatagaagcgacctctggaaaccccatcagggtgcccactggatgctcaatagctgagttttgctcgcctttgatgataccatgtccttgccactcttatttgagtgagaatgtgtcagaagtcatttgttacagtccaaagtggaccaggcctgtcactctagagtataacaactccatatcttggtacccctatacaatccctggtgcgaggggatgtatggttaaattcaaaaataacacatggggttgttgccgtattcgcaatgtgccatcgtactgcactatgggcactgatgcagtgtggaacgacactcgcaacacttacgaagcatgcggtgtaacaccatggctaacaaccgcatggcacaacggctcagccctgaaattggctatattacaataccctgggtctaaagaaatgtttaaacctcataattggatgtcaggccatttgtattttgagggatcagatacccctatagtttacttttatgaccctgtgaattccactctcctaccaccggagaggtgggctaggttgcccggtaccccacctgtggtacgtggttcttggttacaggttccgcaagggttttacagtgatgtgaaagacctagccacaggattgatcaccaaagacaaagcctggaaaaattatcaggtcttatattccgccacgggtgctttgtctcttacgggagttaccaccaaggccgtggtgctaattctgttggggttgtgtggcagcaagtatcttattttagcctacctctgttacttgtccctttgttttgggcgcgcttctggttaccctttgcgtcctgtgctcccatcccagtcgtatctccaagctggctgggatgttttgtctaaagctcaagtagctccttttgctttgattttcttcatctgttgctatctccgctgcaggctacgttatgctgcccttttagggtttgtgcccatggctgcgggcttgcccctaactttctttgttgcagcagctgctgcccaaccagattatgactggtgggtgcgactgctagtggcagggttagttttgtgggccggccgtgaccgtggtcaccgcatagctctgcttgtaggtccttggcctctggtagcgcttttaaccctcttgcatttggttacgcctgcttcagcttttgacaccgagataattggagggctgacaataccacctgtagtagcattagttgtcatgtctcgttttggcttctttgctcacttgttacctcgctgtgctttagttaactcctatctttggcaacgttgggagaattggttttggaacgttacactaagaccggagaggttttttcttgtgctggtttgtttccccggtgcgacatatgacgcgctggtgactttctgtgtgtgtcacgtagctctcctatgtttaacatccagtgcagcatcgttctttgggactgactctagggttagggcccatagaatgttggtgcgtctcggaaagtgccatgcttggtattctcattatgttcttaagtttttcctcttagtgtttggtgagaatggtgtgtttttctataagcacttgcatggtgatgtcttgcctaatgattttgcctcgaaactaccattgcaagagccatttttcccttttgaaggcaaggcaagggtctataggaatgaaggaagacgcttggcgtgtggggacacggttgatggtttgcccgttgttgcgcgtctcggcgaccttgttttcgcagggttagctatgccgccagatgggtgggccattaccgcaccttttacgctgcagtgtctctctgaacgtggcacgctgtcagcgatggcagtggtcatgactggtatagacccccgaacttggactggaactatcttcagattaggatctctggccactagctacatgggatttgtttgtgacaacgtgttgtatactgctcaccatggcagcaaggggcgccggttggctcatcccacaggctccatacacccaataaccgttgacgcggctaatgaccaggacatctatcaaccaccatgtggagctgggtcccttactcggtgctcttgcggggagaccaaggggtatctggtaacacgactggggtcattggttgaggtcaataaatccgatgacccttattggtgtgtgtgcggggcccttcccatggctgttgccaagggttcttcaggtgccccgattctgtgctcctccgggcatgttattgggatgttcaccgctgctagaaattctggcggttcagtcagccagattagggttaggccgttggtgtgtgctggataccatccccagtacacagcacatgccactcttgatacaaaacctactgtgcctaacgagtattcagtgcaaattttaattgcccccactggcagcggcaagtcaaccaaattaccactttcttacatgcaggagaagtatgaggtcttggtcctaaatcccagtgtggctacaacagcatcaatgccaaagtacatgcacgcgacgtacggcgtgaatccaaattgctattttaatggcaaatgtaccaacacaggggcttcacttacgtacagcacatatggcatgtacctgaccggagcatgttcccggaactatgatgtaatcatttgtgacgaatgccatgctaccgatgcaaccaccgtgttgggcattggaaaggtcctaaccgaagctccatccaaaaatgttaggctagtggttcttgccacggctaccccccctggagtaatccctacaccacatgccaacataactgagattcaattaaccgatgaaggcactatcccctttcatggaaaaaagattaaggaggaaaatctgaagaaagggagacaccttatctttgaggctaccaaaaaacactgtgatgagcttgctaacgagttagctcgaaagggaataacagctgtctcttactataggggatgtgacatctcaaaaatccctgagggcgactgtgtagtagttgccactgatgccttgtgtacagggtacactggtgactttgattccgtgtatgactgcagcctcatggtagaaggcacatgccatgttgaccttgaccctactttcaccatgggtgttcgtgtgtgcggggtttcagcaatagttaaaggccagcgtaggggccgcacaggccgtgggagagctggcatatactactatgtagacgggagttgtaccccttcgggtatggttcctgaatgcaacattgttgaagccttcgacgcagccaaggcatggtatggtttgtcatcaacagaagctcaaactattctggacacctatcgcacccaacctgggttacctgcgataggagcaaatttggacgagtgggctgatctcttttcaatggtcaaccccgaaccttcatttgtcaatactgcaaaaagaactgctgacaattatgttttgttgactgcagcccaactacaactgtgtcatcagtatggctatgctgctcccaatgacgcaccacggtggcagggagcccggcttgggaaaaaaccttgtggggttctgtggcgcttggacggcgctgacgcctgtcctggcccagagcccagcgaggtgaccagataccaaatgtgcttcactgaagtcaatacttctgggacagccgcactcgctgttggcgttggagtggctatggcttatctagccattgacacttttggcgccacttgtgtgcggcgttgctggtctattacatcagtccctaccggtgctactgtcgccccagtggttgacgaagaagaaatcgtggaggagtgtgcatcattcattcccttggaggccatggttgctgcaattgacaagctgaagagtacaatcaccacaactagtcctttcacattggaaaccgcccttgaaaaacttaacacctttcttgggcctcatgcagctacaatccttgctatcatagagtattgctgtggcttagtcactttacctgacaatccctttgcatcatgcgtgtttgctttcattgcgggtattactaccccactacctcacaagatcaaaatgttcctgtcattatttggaggcgcaattgcgtccaagcttacagacgctagaggcgcactggcgttcatgatggccggggctgcgggaacagctcttggtacatggacatcggtgggttttgtctttgacatgctaggcggctatgctgccgcctcatccactgcttgcttgacatttaaatgcttgatgggtgagtggcccactatggatcagcttgctggtttagtctactccgcgttcaacccggccgcaggagttgtgggcgttttgtcagcttgtgcaatgtttgctttgacaacagcagggccagatcactggcccaacagacttcttactatgcttgctaggagcaacactgtatgtaatgagtactttattgccactcgtgacatccgcaggaagatactgggcattctggaggcatctaccccctggagtgtcatatcagcttgcatccgttggctccacaccccgacggaggatgattgcggcctcattgcttggggtctagagatttggcagtacgtgtgcaatttctttgtgatttgctttaatgtccttaaagctggagttcagagcatggttaacattcctggttgtcctttctacagctgccagaaggggtacaagggcccctggattggatcaggtatgctccaagcacgctgtccatgcggtgctgaactcatcttttctgttgagaatggttttgcaaaactttacaaaggacccagaacttgttcaaattactggagaggggctgttccagtcaacgctaggctgtgtgggtcggctagaccggacccaactgattggactagtcttgtcgtcaattatggcgttagggactactgtaaatatgagaaattgggagatcacatctttgttacagcagtatcctctccaaatgtctgtttcacccaggtgcccccaaccttgagagctgcagtggccgtggacggcgtacaggttcagtgttatctaggtgagcccaaaactccttggacgacatctgcttgctgttacggtcctgacggtaagggtaaaactgttaagcttcccttccgcgttgacggtcacacacctggtgtgcgcatgcaacttaatttgcgtgatgcacttgagacaaatgactgtaattccacaaacaacactcctagtgatgaagccgcagtgtccgctcttgttttcaaacaggagttgcggcgtacaaaccaattgcttgaggcaatttcagctggcgttgacaccaccaaactgccagccccctccatcgaagaggtagtggtaagaaagcgccagttccgggcaagaactggttcgcttaccttgcccccccctccgagatccgtcccaggagtgtcatgtcctgaaagcctgcaacgaagtgacccgttagaaggtccttcaaacctccctccttcaccacctgttctacagttggccatgccgatgcccctgttgggagcgggtgagtgtaaccctttcactgcaattggatgtgcaatgaccgaaacaggcggaggccctgatgatttacccagttaccctcccaaaaaggaggtctctgaatggtcagacgaaagttggtcaacggctacaaccgcttccagctacgttactggccccccgtaccctaagatacggggaaaggattccactcagtcagcccccgccaaacggcctacaaaaaagaagttgggaaagagtgagttttcgtgcagcatgagctacacttggaccgacgtgattagcttcaaaactgcttctaaagttctgtctgcaactcgggccatcactagtggtttcctcaaacaaagatcattggtgtatgtgactgagccgcgggatgcggagcttagaaaacaaaaagtcactattaatagacaacctctgttccccccatcataccacaagcaagtgagattggctaaggagaaagcttcaaaagttgtcggtgtcatgtgggactatgatgaagtagcagctcacacgccctctaagtctgctaagtcccacatcactggccttcggggcactgatgttcgttctggagcagcccgcaaggctgttctggacttgcagaagtgtgtcgaggcaggtgagataccgagtcattatcggcaaacagtgatagttccaaaggaggaggtcttcgtgaagaccccccagaaaccaacaaagaaacccccaaggctcatctcgtacccccaccttgaaatgagatgtgttgagaagatgtactacggtcaggttgctcctgacgtagttaaagctgtcatgggagatgcgtacgggtttgtagatccacgtacccgtgtcaagcgtctgttgtcgatgtggtcacccgatgcagtcggagccacatgcgatacagtgtgttttgacagtaccatcacacccgaggatatcatggtggagacagacatctactcagcagctaaactcagtgaccaacaccgagctggcattcacaccattgcgaggcagttatacgctggaggaccgatgatcgcttatgatggccgagagatcggatatcgtaggtgtaggtcttccggcgtctatactacctcaagttccaacagtttgacctgctggctgaaggtaaatgctgcagccgaacaggctggcatgaagaaccctcgcttccttatttgcggcgatgattgcaccgtaatttggaaaagcgccggagcagatgcagacaaacaagcaatgcgtgtctttgctagctggatgaaggtgatgggtgcaccacaagattgtgtgcctcaacccaaatacagtttggaagaattaacatcatgctcatcaaatgttacctctggaattaccaaaagtggcaagccttactactttcttacaagagatcctcgtatcccccttggcaggtgctctgccgagggtctgggatacaaccccagtgctgcgtggattgggtatctaatacatcactacccatgtttgtgggttagccgtgtgttggctgtccatttcatggagcagatgctctttgaggacaaacttcccgagactgtgacctttgactggtatgggaaaaattatacggtgcctgtagaagatctgcccagcatcattgctggtgtgcacggtattgaggctttctcggtggtgcgctacaccaacgctgagatcctcagagtttcccaatcactaacagacatgaccatgccccccctgcgagcctggcgaaagaaagccagggcggtcctcgccagcgccaagaggcgtggcggagcacacgcaaaattggctcgcttccttctctggcatgctacatctagacctctaccagatttggataagacgagcgtggctcggtacaccactttcaattattgtgatgtttactccccggagggggatgtgtttgttacaccacagagaagattgcagaagtttcttgtgaagtatttggctgtcattgtttttgccctagggctcattgctgttggattagccatcagctgaacccccaaattcaaaattaactaacagtttttttttttttttttttttagggcagcggcaacaggggagaccccgggcttaacgaccccgccgatgtgagtttggcgaccatggtggatcagaaccgtttcgggtgaagccatggtctgaaggggatgacgtcccttctggctcatccacaaaaaccgtctcgggtgggtgaggagtcctggctgtgtgggaagcagtcagtataattcccgtcgtgtgtggtgacgcctcacgacgtacttgtccgctgtgcagagcgtagtaccaagggctgcaccccggtttttgttccaagcggagggcaacccccgcttggaattaaaaact

SEQ ID NO: 3 - protein, 2864 aa, Artificial Sequence
  GBV-B Replicon
MPVISTQTSPVPAPRTRKNKQTQASYPVSIKTSVERGQRAKRKVQRDARPRNYKIAGIHDGLQTLAQAALPAHGWGRQDPRHKSRNLGILLDYPLGWIGDVTTHTPLVGPLVAGAVVRPVCQIVRLLEDGVNWATGWFGVHLFVVCLLSLACPCSGARVTDPDTNTTILTNCCQRNQVIYCSPSTCLHEPGCVICADECWVPANPYISHPSNWTGTDSFLADHIDFVMGALVTCDALDIGELCGACVLVGDWLVRHWLIHIDLNETGTCYLEVPTGIDPGFLGFIGWMAGKVEAVIFLTKLASQVPYAIATMFSSVHYLAVGALIYYASRGKWYQLLLALMLYIEATSGNPIRVPTGCSIAEFCSPLMIPCPCHSYLSENVSEVICYSPKWTRPVTLEYNNSISWYPYTIPGARGCMVKFKNNTWGCCRIRNVPSYCTMGTDAVWNDTRNTYEACGVTPWLTTAWHNGSALKLAILQYPGSKEMFKPHNWMSGHLYFEGSDTPIVYFYDPVNSTLLPPERWARLPGTPPVVRGSWLQVPQGFYSDVKDLATGLITKDKAWKNYQVLYSATGALSLTGVTTKAVVLILLGLCGSKYLILAYLCYLSLCFGRASGYPLRPVLPSQSYLQAGWDVLSKAQVAPFALIFFICCYLRCRLRYAALLGFVPMAAGLPLTFFVAAAAAQPDYDWWVRLLVAGLVLWAGRDRGHRIALLVGPWPLVALLTLLHLVTPASAFDTEIIGGLTIPPVVALVVMSRFGFFAHLLPRCALVNSYLWQRWENWFWNVTLRPERFFLVLVCFPGATYDALVTFCVCHVALLCLTSSAASFFGTDSRVRAHRMLVRLGKCHAWYSHYVLKFFLLVFGENGVFFYKHLHGDVLPNDFASKLPLQEPFFPFEGKARVYRNEGRRLACGDTVDGLPVVARLGDLVFAGLAMPPDGWAITAPFTLQCLSERGTLSAMAVVMTGIDPRTWTGTIFRLGSLATSYMGFVCDNVLYTAHHGSKGRRLAHPTGSIHPITVDAANDQDIYQPPCGAGSLTRCSCGETKGYLVTRLGSLVEVNKSDDPYWCVCGALPMAVAKGSSGAPILCSSGHVIGMFTAARNSGGSVSQIRVRPLVCAGYHPQYTAHATLDTKPTVPNEYSVQILIAPTGSGKSTKLPLSYMQEKYEVLVLNPSVATTASMPKYMHATYGVNPNCYFNGKCTNTGASLTYSTYGMYLTGACSRNYDVIICDECHATDATTVLGIGKVLTEAPSKNVRLVVLATATPPGVIPTPHANITEIQLTDEGTIPFHGKKIKEENLKKGRHLIFEATKKHCDELANELARKGITAVSYYRGCDISKIPEGDCVVVATDALCTGYTGDFDSVYDCSLMVEGTCHVDLDPTFTMGVRVCGVSAIVKGQRRGRTGRGRAGIYYYVDGSCTPSGMVPECNIVEAFDAAKAWYGLSSTEAQTILDTYRTQPGLPAIGANLDEWADLFSMVNPEPSFVNTAKRTADNYVLLTAAQLQLCHQYGYAAPNDAPRWQGARLGKKPCGVLWRLDGADACPGPEPSEVTRYQMCFTEVNTSGTAALAVGVGVAMAYLAIDTFGATCVRRCWSITSVPTGATVAPVVDEEEIVEECASFIPLEAMVAAIDKLKSTITTTSPFTLETALEKLNTFLGPHAATILAIIEYCCGLVTLPDNPFASCVFAFIAGITTPLPHKIKMFLSLFGGAIASKLTDARGALAFMMAGAAGTALGTWTSVGFVFDMLGGYAAASSTACLTFKCLMGEWPTMDQLAGLVYSAFNPAAGVVGVLSACAMFALTTAGPDHWPNRLLTMLARSNTVCNEYFIATRDIRRKILGILEASTPWSVISACIRWLHTPTEDDCGLIAWGLEIWQYVCNFFVICFNVLKAGVQSMVNIPGCPFYSCQKGYKGPWIGSGMLQARCPCGAELIFSVENGFAKLYKGPRTCSNYWRGAVPVNARLCGSARPDPTDWTSLVVNYGVRDYCKYEKLGDHIFVTAVSSPNVCFTQVPPTLRAAVAVDGVQVQCYLGEPKTPWTTSACCYGPDGKGKTVKLPFRVDGHTPGVRMQLNLRDALETNDCNSTNNTPSDEAAVSALVFKQELRRTNQLLEAISAGVDTTKLPAPSIEEVVVRKRQFRARTGSLTLPPPPRSVPGVSCPESLQRSDPLEGPSNLPPSPPVLQLAMPMPLLGAGECNPFTAIGCAMTETGGGPDDLPSYPPKKEVSEWSDESWSTATTASSYVTGPPYPKIRGKDSTQSAPAKRPTKKKLGKSEFSCSMSYTWTDVISFKTASKVLSATRAITSGFLKQRSLVYVTEPRDAELRKQKVTINRQPLFPPSYHKQVRLAKEKASKVVGVMWDYDEVAAHTPSKSAKSHITGLRGTDVRSGAARKAVLDLQKCVEAGEIPSHYRQTVIVPKEEVFVKTPQKPTKKPPRLISYPHLEMRCVEKMYYGQVAPDVVKAVMGDAYGFVDPRTRVKRLLSMWSPDAVGATCDTVCFDSTITPEDIMVETDIYSAAKLSDQHRAGIHTIARQLYAGGPMIAYDGREIGYRRCRSSGVYTTSSSNSLTCWLKVNAAAEQAGMKNPRFLICGDDCTVIWKSAGADADKQAMRVFASWMKVMGAPQDCVPQPKYSLEELTSCSSNVTSGITKSGKPYYFLTRDPRIPLGRCSAEGLGYNPSAAWIGYLIHHYPCLWVSRVLAVHFMEQMLFEDKLPETVTFDWYGKNYTVPVEDLPSIIAGVHGIEAFSVVRYTNAEILRVSQSLTDMTMPPLRAWRKKARAVLASAKRRGGAHAKLARFLLWHATSRPLPDLDKTSVARYTTFNYCDVYSPEGDVFVTPQRRLQKFLVKYLAVIVFALGLIAVGLAIS

SEQ ID NO: 4 - DNA, 35 bp, Artificial Sequence
  Partial GBV-B Replicon Sequence
gaccgtagcacatggggcgcgccatgattgaacaa

SEQ ID NO: 5 - DNA, 56 bp, Artificial Sequence
  Partial GBV-B Replicon Sequence
gaccgtagcacatgcctgttatttctactcaaacagggcgcgccatgattgaacaa

SEQ ID NO: 6 - DNA, 74 bp, Artificial Sequence
  Partial GBV-B Replicon Sequence
gaccgtagcacatgcctgttatttctactcaaacaagtcctgtacctgcgcccgggcgcgccatgattgaacaa

SEQ ID NO: 7 - DNA, 98 bp, Artificial Sequence
  Partial GBV-B Replicon Sequence
gaccgtagcacatgcctgttatttctactcaaacaagtcctgtacctgcgcccagaacgcgcaagaacaagcagacggggcgcgccatgattgaacaa

SEQ ID NO: 8 - protein, 8 aa, Artificial Sequence
  Partial GBV-B Replicon Sequence
MGRAMIEQ

SEQ ID NO: 9 - protein, 15 aa, Artificial Sequence
  Partial GBV-B Replicon Sequence
MPVISTQTGRAMIEQ

SEQ ID NO: 10 - protein, 21 aa, Artificial Sequence
  Partial GBV-B Replicon Sequence
MPVISTQTSPVPAPGRAMIEQ

SEQ ID NO: 11 - protein, 29 aa, Artificial Sequence
  Partial GBV-B Replicon Sequence
MPVISTQTSPVPAPRTRKNKQTGRAMIEQ

SEQ ID NO: 12 - protein, 291 aa, Artificial Sequence
  Partial HCV Replicon Sequence
GHAVGIFRAAVCTRGVAKAVDFVPVESMXTTMRSPVFTDNSSPPAVPQTFQVAHLHAPTGSGKSTKVPAAYAAQGYKVLVLNPSVAATLGFGAYMSKAHGIDPNIRXGVRTITTGAPLTSMLTXPSHITAETAKRXLARGSXXSLXSSSAXQLSAPSLKATCTTRHDSPDADLIEANLLWRQEMGGNITRVESENKVVILDSFEPLQAEEDEREVSVPAEILRRSRKFPRAYSIEPLDLPQIIQXLHGLSAFSLHSYSPGEINRVASCLRKLGVPPLRVWRHRARSVRARL

SEQ ID NO: 13 - protein, 270 aa, Artificial Sequence
  Partial GBV-B Replicon Sequence
GHVIGMFTAARNSGGSVSQIRVRPLVCAGYHPQYTAHATLDTKPTVPNEYSVQILIAPTGSGKSTKLPLSYMQEKYEVLVLNPSVATTASMPKYMHATYGVNPNCYFNGKCTNTGASKTVKLPFRVDGHTPGVRMQLNLRDALETNDCNSTNNTPSDEAAVSALVFKQELRRTNQLLEAISAGVDTTKLPAPSIEEVVVRKRQFRARTGSYTVPVEDLPSIIAGVHGIEAFSVVRYTNAEILRVSQSLTDMTMPPLRAWRKKARAVLASA

SEQ ID NO: 14 - DNA, 18 bp, Artificial Sequence
  Oligonucleotide Primer
gtaggcggcgggactcat

SEQ ID NO: 15 - DNA, 19 bp, Artificial Sequence
  Oligonucleotide Primer
tcagggccatccaagtcaa

SEQ ID NO: 16 - DNA, 22 bp, Artificial Sequence
  Oligonucleotide Probe
tcgcgtgatgacaagcgccaag

SEQ ID NO: 17 - DNA, 19 bp, Artificial Sequence
  Oligonucelotide Primer
gatggattgcacgcaggtt

SEQ ID NO: 18 - DNA, 21 bp, Artificial Sequence
  Oligonucleotide Primer
cccagtcatagccgaatagcc

SEQ ID NO: 19 - DNA, 19 bp, Artificial Sequence
  Oligonucleotide Probe
tccggccgcttgggtggag

(19) United States
(12) Patent Application Publication
(10) Pub. No.: US 2005/0239205 A1
(43) Pub. Date: Oct. 27, 2005

(54) Gb virus b based replicons and replicon enhanced cells

(76) Inventors: Amedeo De Tomassi, Rome (IT); Rita Graziani, Rome (IT); Giacomo Paonessa, Rome (IT); Cinzia Traboni, Rome (IT)

Correspondence Address: MERCK AND CO., INC, P O BOX 2000, RAHWAY, NJ, 07065-0907, US

(21) Appl. No.: 10/501,412

(22) PCT Filed: Jan. 13, 2003
(86) PCT No.: PCT/EP03/00281

§ 371 Date: Jul. 12, 2004

Related U.S. Application Data
(60) Provisional application No. 60/348,573, filed on Jan. 15, 2002.
(60) Provisional application No. 60/386,655, filed on Jun. 6, 2002.

(51) Int. Cl. C12N 7/00; C12N 7/01; C12N 5/06; C12N 15/86

(52) U.S. Cl. 435/456; 435/235.1; 435/364

(57) ABSTRACT

The present invention features GBV-B replicons and replicon enhanced cells. A GBV-B replicon is an RNA molecule able to autonomously replicate in a cultured cell and produce detectable levels of one or more GBV-B proteins. GBV-B replicon enhanced cells are cells having an increased ability to maintain a GBV-B replicon.

### CROSS-REFERENCE TO RELATED APPLICATIONS

The present application claims priority to provisional application U.S. Ser. No. 60/386,655, filed Jun. 6, 2002, and provisional application U.S. Ser. No. 60/348,573, filed Jan. 15, 2002, hereby incorporated by reference herein.

### BACKGROUND OF THE INVENTION

The references cited throughout the present application are not admitted to be prior art to the claimed invention.

It is estimated that about 3% of the world's population is infected with the hepatitis C virus (HCV). (Wasley et al., Semin. Liver Dis. 20:1-16, 2000.) HCV exposure results in an overt acute disease in a small percentage of cases, while in most instances the virus establishes a chronic infection causing liver inflammation and slowly progresses into liver failure and cirrhosis. (Strader et al., ILAR J. 42:107-116, 2001.) Epidemiological surveys indicate an important role for HCV in the onset of hepatocellular carcinoma. (Strader et al., ILAR J. 42:107-116, 2001).

Investigating the effects of HCV and antiviral compounds is complicated by the absence of a small animal model. HCV infects human and chimpanzees, but does not infect small animals such as mice and rats.

The GB virus B (GBV-B) can infect different new world monkeys such as tamarins and owl monkeys. (Bukh et al., Journal of Medical Virology 65:694-697, 2001.) GBV-B has been proposed as a surrogate model for studying HCV and the effects of antiviral compounds. (Traboni, International Publication Number WO/73466, International Publication Date 7 Dec. 2000, Bukh et al., International Publication Number WO/75337, International Publication Date 14 Dec. 2000).

The hypothesis of deriving information useful for research of anti-HCV drugs from experiments with GBV-B in tamarins has been supported by data concerning enzymatic activity of viral proteins and the role of untranslated regions, as well as the identification of in vivo infectious cDNA and the establishment of a cell-based infection system. (See for example, Beames et al., J. Virol. 74:11764-11772, 2000, Traboni et al., The GB viruses: a comprehensive survey. In S. G. Pandalai (ed.), Recent Research Developments in Virology, part III. Transworld Research Network, 1999.)

The similarity between HCV and GBV-B genome organization was underlined since GBV-B was discovered in 1995. (Muerhoff et al., J. Virol. 69:5621-5630, 1995.) Early experimental demonstration of the similarity at the functional level came from the enzymatic activity of NS3 protease. (Scarselli et al., J. Virol. 71:49854989, 1997.) Subsequent analyses have been performed looking at different HCV and GBV-B regions.

Studies performed examining polyprotein processing and the functional relationship between the HCV and GBV-B NS3 proteins indicate overlapping specificity, and a virus-specific NS4A cofactor requirement. (Butkiewicz et al., J. Virol. 74:4291-4301, 2000, Sbardellati et al., J. Gen. Virol. 81 Pt 9:2183-2188, 2000.)

The helicase and NTP-ase activity associated with the C-terminal domain of GBV-B NS3 protein has been reported as comparable to those of HCV. (Zhong et al., Virology. 261:216-226, 1999.) The RNA-dependent RNA polymerase activity encoded by a truncated form of GBV-B NS5B and HCV NS5B showed similarities. (Zhong et al., J. Viral Hepat. 7:335-342, 2000).

The HCV and GBV-B 5′ and 3′ untranslated regions play an important role in the initiation of the replication process via interactions with viral proteins such as helicase and RNA-dependent RNA polymerase. The HCV and GBV-B 5′ and 3′ untranslated regions contain common features. The internal ribosome entry site containing 5′-UTR of GBV-B shows both structural and functional similarity to that of HCV. (Grace et al., J. Gen. Virol. 80:2337-2341, 1999, Rijnbrand et al., J. Virol. 74:773-783, 2000, Rijnbrand et al., Rna. 7:585-597, 2001.) The 3′end of the GBV-B 3′UTR is arranged in a similar secondary structure to HCV and is important for replication and in vivo infectivity. (Bukh et al., Virology 262:470-478, 1999, Sbardellati et al., Journal of Virology 73:10546-10550, 1999, Sbardellati et al., J. Gen. Virol. 82:2437-2448, 2001.)

### SUMMARY OF THE INVENTION

The present invention features GBV-B replicons and replicon enhanced cells. A GBV-B replicon is an RNA molecule able to autonomously replicate in a cultured cell and produce detectable levels of one or more GBV-B proteins. GBV-B replicon enhanced cells are cells having an increased ability to maintain a GBV-B replicon.

Functional GBV-B genomic and subgenomic replicons can be obtained based on GBV-B sequences such as those provided in SEQ ID NO: 1 and SEQ. ID. NO. 2. SEQ. ID. NO. 1 provides a bicistronic subgenomic replicon sequence illustrated herein as able to replicate in a cell. SEQ. ID. NO. 2 provides the cDNA sequence of a genomic GBV-B replicon infectious in tamarins.

GBV-B replicon enhanced cells can be produced by selecting for a cell able to maintain a GBV-B replicon and curing the cell of the replicon. The replicon enhanced cell has an increased ability to maintain a replicon upon subsequent transfection. The replicon used in the subsequent transfection can be different from the replicon used to produce the replicon enhanced cell. For example, a bicistronic GBV-B replicon with a selection/reporter sequence can be used to obtain the enhanced cell and a second replicon without such a selection/reporter sequence, including a full-length infectious replicon, can be introduced into the replicon enhanced cell.

Thus, a first aspect of the present invention describes a GBV-B replicon capable of replication in a cell comprising the following regions:
a GBV-B 5′ UTR substantially similar to bases 1-445 of SEQ. ID. NO. 1;
a selection or reporter sequence functionally coupled to the GBV-B 5′ UTR;
an internal ribosome entry site;
a NS3-NS5B sequence substantially similar to bases 1938-7709 of SEQ. ID. NO. 1 functionally coupled to the internal ribosome entry site and an AUG translation initiation codon; and
a GBV-B 3′ UTR substantially similar to bases 7710-8069 of SEQ. ID. NO. 1.

Reference to “comprising the following regions” indicates that the provided regions are present and additional regions may also be present. The additional regions are preferably located in a position between the 5′ GBV-B UTR and GBV-B 3′ UTR. However, the additional region can be, for example, an additional cistron located 5′ of the 5′ GBV-B UTR.

Preferred additional regions are GBV-B structural region(s) and the GBV-B NS2 region. Additional regions can be of different sizes such as a partial core region or a complete structural GBV-B region and can be provided together in one region. For example, the NS2 region can be provided at the 3′ end of a structural region.

Additional regions can be present in different replicon locations. Examples of different locations include providing a structural region and/or NS2 region in a cistron containing the GBV-B 5′ UTR and providing a structural region and/or NS2 region in a cistron comprising NS3-NSSB.

Reference to “functionally coupled” indicates the ability of a first nucleotide sequence to mediate an effect on a second nucleotide sequence. Functionally coupled does not require that the coupled sequences be adjacent to each other. A GBV-B 5′-UTR and an internal ribosome entry site facilitates ribosome binding and/or translation of the sequences to which they are coupled. The GBV-B 3′ UTR is important for replicon replication.

Another aspect of the present invention describes an expression vector comprising a promoter transcriptionally coupled to a nucleotide sequence coding for a GBV-B replicon described herein.

Another aspect of the present invention describes a GBV-B replicon made by a process comprising the steps of transfecting a cell with a GBV-B replicon and isolating the replicon.

Another aspect of the present invention describes a method of making a second GBV-B replicon from a first GBV replicon comprising the steps of: (a) transfecting a cell with the first replicon; (b) isolating a replicon from the transfected cell; (c) determining the nucleotide sequence of the replicon from the transfected cell; and (d) producing the second GBV-B replicon, wherein the second replicon contains the first replicon sequence with one or more alterations corresponding to the transfected cell replicon sequence. Preferably, the second replicon has the same sequence as the transfected cell replicon sequence.

Another aspect of the present invention describes a method of measuring the ability of a compound to affect GBV-B replicon activity. The method involves providing the compound to a cell containing a GBV-B replicon and measuring the ability of the compound to affect one or more replicon activities as a measure of the effect on GBV-B activity.

Another aspect of the present invention describes a GBV-B replicon enhanced cell wherein the cell has a maintenance and activity efficiency of at least 25% when transfected with a GBV-B replicon of SEQ ID. NO. 1 by the Electroporation Method.

Reference to the “Electroporation Method” indicates the transfection techniques described in Example 1 infra. The replicon enhanced cells need not be produced from a particular cell type or by a particular technique, but rather has as a property a maintenance and activity efficiency of at least 25% upon transfection of the GBV-B replicon of SEQ. ID. NO. 1 using the Electroporation Method.

A maintenance and activity efficiency of at least 25% indicates that at least 25% of the cells used in the Electroporation Method maintain functional replicons. In different embodiments the maintenance and activity efficiency is at least 35%, at least 50%, or at least 75%.

Preferred replicon enhanced cells are Huh7 or Hep3B derived cells. “Derived cells” are cells produced starting with a particular cell (e.g., Huh7 or Hep3B) and selecting, introducing or producing one or more phenotypic or genotypic modifications.

Another aspect of the present invention describes a method of making a GBV-B replicon enhanced cell. The method involves the steps of: (a) introducing and maintaining a GBV-B replicon into a cell and (b) curing the cell of the replicon.

Another aspect of the present invention describes a GBV-B replicon enhanced cell made by a process comprising the steps of: (a) introducing and maintaining a GBV-B replicon into a cell and (b) curing the cell of the replicon.

Another aspect of the present invention describes a method of making a GBV-B replicon enhanced cell comprising a GBV-B replicon. The method involves producing a replicon enhanced cell and introducing and maintaining a GBV-B replicon in the cell.

Another aspect of the present invention describes a GBV-B replicon enhanced cell containing a GBV-B replicon made by a process involving producing a replicon enhanced cell and introducing and maintaining the GBV-B replicon in the cell.

Another aspect of the present invention describes a method of measuring the ability of a compound to affect GBV-B replicon activity using a GBV-B replicon enhanced cell comprising a GBV-B replicon. The method involves providing a compound to the cell and measuring the ability of the compound to affect one or more replicon activities as a measure of the effect on GBV-B replicon activity.

Another aspect of the present invention describes a method of producing an infectious GBV-B virion. The method comprises the steps of growing a replicon enhanced cell containing a replicon encoding a GB V-B virion to produce the GBV-B virion.

Another aspect of the present invention describes a method of infecting an animal with a GBV-B virion. The method involves producing the virion and providing the virion to an animal.

Another aspect of the present invention describes a method for producing a chimeric GBV-B/HCV replicon. The method involves the step of replacing one or more GBV-B regions or portion thereof present in a replicon described herein with the corresponding region from HCV.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A-1F provide the neo-RepD replicon sequence (SEQ. ID. NO. 1). Nucleotide number 1 is the first of GBV-B genome. Core region is in capital letters. The approximate location of the GBV-B regions are provided as follows:
1-445: GBV-B 5′ non-translated region, drives translation of the core-neo fusion protein;
446-1315 (including stop codon): core-neo fusion protein, selectable marker;
1324-1934: Internal ribosome entry site of the encephalomyocarditis virus, drives translation of the GBV-B NS region;
1935-7709: GBV-B polyprotein from non-structural protein 3 to non-structural protein 5B, including an AUG start codon;
1938-3797 (putative): Non-structural protein 3 (NS3), NS3 protease/helicase;
3798 (putative)-3962: Non-structural protein 4A (NS4A), NS3 protease cofactor;
3963-4706 (putative): Non-structural protein 4B (NS4B);
4707 (putative)-5939: Non-structural protein 5A (NS5A);
5940-7709 (excluding stop codon): Non-structural protein 5B (NS5B); GBV-B RNA-dependent RNA polymerase; and
7710-8069: GBV-B 3′ non-translated region.

FIGS. 2A-2G illustrate the cDNA (SEQ. ID. NO. 2) for a full-length GBV-B replicon sequence infectious in tamarins (Sbardellati et al., J. Gen. Virol. 82:2437-2448, 2001). The approximate location of the GBV-B regions are provided as follows:
1-445: GBV-B 5′ non-translated region, drives translation of the GBV-B polyprotein;
446-9037: GBV-B polyprotein from core protein to non-structural protein 5B;
446-919 (putative): structural protein core, nucleocapsid protein;
920 (putative)-1489 (putative): structural protein E1, envelope protein;
1490 (putative)-2641 (putative):structural protein E2, envelope protein;
2642 (putative)-3265: Non-structural protein 2 (NS2);
3266-5125 (putative): Non-structural protein 3 (NS3), GBV-B NS3 protease/helicase;
5126 (putative)-5289: Non-structural protein 4A (NS4A), NS3 protease cofactor;
5290-6034 (putative): Non-structural protein 4B (NS4B);
6035 (putative)-7267: Non-structural protein 5A (NSSA);
7268-9037 (excluding stop codon): Non-structural protein 5B (NS5B); GBV-B RNA-dependent RNA polymerase; and
9038-9397: GBV-B 3′ non-translated region.

FIGS. 3A-3C illustrates the amino acid sequence (SEQ. ID. NO. 3) for a full-length GBV-B replicon sequence infectious in tamarins (Sbardellati et al., J. Gen. Virol. 82:2437-2448, 2001). The approximate location of the GBV-B regions are provided as follows:
1-158 (putative): structural protein core, nucleocapsid protein;
159 (putative)-348 (putative): structural protein E1, envelope protein;
349 (putative)-732 (putative): structural protein E2, envelope protein;
733 (putative)-940: Non-structural protein 2 (NS2);
941-1560 (putative): Non-structural protein 3 (NS3), GBV-B NS3 protease/helicase;
1561 (putative)-1615: Non-structural protein 4A (NS4A), NS3 protease cofactor;
1616-1863 (putative): Non-structural protein 4B (NS4B);
1864 (putative)-2274: Non-structural protein 5A (NSSA); and
2275-2864: Non-structural protein 5B (NSSB); GBV-B RNA-dependent RNA polymerase.

FIG. 4 provides a schematic representation of the GBV-B neo-RepA, neo-RepB, neo-RepC and neo-RepD constructs. The nucleotide sequences below the drawing correspond to the 3′end of the GBV-B 5′UTR, the partial core coding sequence, the nucleotides added to create a restriction site and to put the subsequent neomycin phosphotransferase gene sequences in the same translation frame of partial core sequence, and the 5′-end of neomycin phosphotransferase gene sequences. The above described sequences corresponding to neo-RepA, neo-RepB, neo-RepC and neo-RepD constructs respectively are shown as SEQ. ID. NOs. 4, 5, 6 and 7. The portion of the sequence belonging to GBV-B 5′-UTR is underlined, that representing translated GBV-B sequences is bold-faced, the sequence corresponding to added PmeI restriction site is in italic and that corresponding to a portion of the neomycin phosphotransferase gene is in regular characters. The translated sequences are organized in nucleotide triplets and the corresponding amino acids are indicated below each cognate nucleotide sequence (SEQ. ID. NOs. 8, 9, 10 and 11).

FIG. 5 illustrates the effects of human α-IFN on the replication of GBV-B and HCV replicons. Clones designated 10A for a HCV replicon in a Huh7 cell and B76.1/Huh7 for a GBV-B replicon in a Huh7 cell were compared. Quantitative PCR was employed using a primer set recognizing the neomycin phosphotransferase gene. Each curve was derived normalizing the replicon RNA amounts to those of endogenous reference GAPDH.

FIG. 6 provides an alignment of deduced amino acid sequences of HCV (SEQ. ID. NO. 12) and GBV-B (SEQ. ID. NO. 13) replicons spanning HCV adaptive mutations. Part of NS3, NS5A and NS5B protein sequences are shown. The position of HCV mutation is underlined in the wild type sequence and the mutated amino acids described in those positions (Bartenschlager et al., Antiviral Res. 52:1-17, 2001) are indicated above the wild type sequence. The line above the HCV NS5A sequence indicates the amino acids missing in a deletion mutant (Blight et al., Science. 290:1972-1974, 2000). Numbering of HCV amino acids is as described in Bartenschlager et al., Antiviral Res. 52:1-17, 2001. The R2884G mutation in NS5B is boldface.

### DETAILED DESCRIPTION OF THE INVENTION

The present invention features GBV-B replicons and replicon enhanced cells. GBV-B replicons and replicon enhanced cells have a variety of uses including: providing tools for studying GBV-B replication, polyprotein production and polyprotein processing; identifying compounds inhibiting GBV-B; providing a surrogate model for identifying compounds inhibiting HCV; and providing a scaffold for producing GBV-B/HCV chimeric replicons.

Compounds inhibiting GBV-B or HCV have research and therapeutic applications. Research applications include using viral inhibitors to study viral proteins, polyprotein processing or viral replication. Therapeutic applications include using those compounds having appropriate pharmacological properties such as efficacy and lack of unacceptable toxicity to treat or inhibit HCV infection in a patient.

The similarities between GBV-B and HCV allow for GBV-B to be used as a surrogate model for testing anti-HCV agents. An advantage of using GBV-B as a surrogate model is its ability to infect animals such as tamarins and owl monkeys. The generally accepted animal model for testing HCV compounds are chimpanzees. Animals susceptible to GBV-B infection such as tamarins and owl monkeys provide a smaller and generally more readily available and less expensive model than chimpanzees.

Using GBV-B replicons and replicon enhanced cells provides an in vitro model that can be used to screen for antiviral compounds prior to infecting an animal susceptible to GBV-B. The animal can then be infected with a GBV-B virus.

### GBV-B Replicons

GBV-B replicons are RNA molecules able to autonomously replicate in a cultured cell and produce detectable levels of one or more GBV-B proteins. GBV-B replicons contain RNA molecules coding for the full-length GBV-B genome or a subgenomic construct. In an embodiment of the present invention the replicon can replicate in a human hepatoma cell, preferably, Huh7 or Hep3B.

A GBV-B replicon may contain non-GBV-B sequences in addition to GBV-B sequences. The additional sequences should not prevent replication and expression, and preferably serve a useful function. Sequences that can be used to serve a useful function include a selection sequence, a reporter sequence, transcription elements and translation elements.

GBV-B Genomic and Subgenomic Regions

GBV-B genomic and subgenomic constructions contain a GBV-B 5′UTR, a GBV-B NS3-NS5B polyprotein encoding region and a GBV-B 3′ UTR. GBV-B genomic constructs also contain a region coding for the structural GBV-B proteins and NS2, while GBV-B subgenomic constructs may also contain all or a portion of the structural region starting at the N-terminal core region and/or NS2. Preferably, the GBV-B genomic construct can produce tamarin infectious GBV-B virions in culture.

The NS3-NS5B polyprotein encoding region provides for a polyprotein that can be processed in a cell into different proteins. Suitable NS3-NS5B polyprotein sequences that may be part of a replicon include those present in different GBV-B strains and functional equivalents thereof resulting in the processing of NS3-NS5B to a produce functional replication machinery. Proper processing can be measured by assaying, for example, NS5B RNA-dependent RNA polymerase, NS3 protease activity or NS3 helicase activity.

The NS3-NS5B polyprotein region also includes either an initial Met translation initiation codon (AUG) or an upstream region able to be translated. An example of such an upstream region is a NS2 region containing a Met translation initiation codon.

The GBV-B 5′ UTR region provides an internal ribosome entry site for protein translation and elements needed for replication. In subgenomic replicons, a partial GBV-B core sequence of at least about 21 nucleotides from the start of the core sequence appears to increase replicon transfection efficiency into cells that are not replicon enhanced. An increase in replicon activity was found to correlate with the length of the partial core sequence inserted before a reporter or selector gene. In different embodiments the partial core sequence is 3′ of an AUG codon and is at least about 21 nucleotides, at least about 39 nucleotides, at least about 63 nucleotides, or about 21-63 nucleotides are present.

In multi-cistronic replicons two or more internal ribosome entry site elements can be present. The internal ribosome entry site towards the 5′ end of the replicon should be a GBV-B 5′ UTR. Additional internal ribosome entry site elements that are present can be non-GBV-B internal ribosome entry site elements. Examples of non-GBV-B internal ribosome entry site elements that can be used are the EMCV internal ribosome entry site, poliovirus internal ribosome entry site, and bovine viral diarrhea virus internal ribosome entry site.

The GBV-B 3′ UTR assists GBV-B replication. GBV-B 3′ UTR includes naturally occurring GBV-B 3′ UTR and functional derivatives thereof. The full length GBV-B 3′ UTR is described by Traboni, International Publication Number WO/73466, International Publication Date 7 Dec. 2000, and Bukh et al., International Publication Number WO/75337, International Publication Date 14 Dec. 2000.

Preferred GBV-B replicons contain a GBV-B sequence able to infect new world monkeys, preferably tamarins and/or owl monkeys. Examples of infectious GBV-B sequences include those provided by Bukh et al., Virology 262:470478, 1999 and Bukh et al., International Publication Number WO/75337, International Publication Date 14 Dec. 2000; and by Sbardellati et al., J. Gen. Virol. 82:2437-2448, 2001, and Traboni, International Publication Number WO/73466, International Publication Date 7 Dec. 2000.

Modifications to an infectious GBV-B replicon sequence can be created using standard techniques to produce, for example, additional infectious GBV-B replicons. Modifications for additional infectious GBV-B replicons provide for one or more nucleic acid substitution(s), insertion(s), deletion(s) or a combination thereof. Different modifications can be designed taking into account nucleic acid sequences and encoded amino acid sequences of different GBV-B sequences; variable and conserved GBV-B amino acid and nucleic acids; and can be experimentally created.

Experimentation to obtain a functional replicon sequence can be performed by introducing a functional replicon into a cell and isolating a replicon from a transfected cell. The spontaneous mutation rate of the replicon RNA sequence will provide different mutations. Those mutations compatible with a functional replicon are selected for by obtaining a replicon expressing cell. The sequence of mutations can be identified and used to produce additional functional replicons.

In different embodiments of the present invention the GBV-B replicon encodes a GBV-B NS3-NS4A-NS4B-NS5A-NS5B sequence substantially similar to residues 941-2864 of SEQ. ID. NO. 3 or a NS2-NS3-NS4A-NS4B-NS5A-NS5B sequence substantially similar to residues 733-2864 of SEQ. ID. NO. 3. Substantially similar amino acid sequences have a sequence identity of at least 85%, at least 95%, at least 99%, or 100%; and/or differ from each other by 1, 2, 3, 4, 5, 6, 7, 8, 9, 10, 11, 12, 13, 14, 15, 16, 17, 18, 19, or 20 amino acids.

Amino acid differences between polypeptides can be calculated by determining the minimum number of amino acid modifications in which the two sequences differ. Amino acid modifications can be deletions, additions, substitutions or any combination thereof.

Amino acid sequence identity can be determined by methods well known in the art that compare the amino acid sequence of one polypeptide to the amino acid sequence of a second polypeptide and generate a sequence alignment. Amino acid identity can be calculated from the alignment by counting the number of aligned residue pairs that have identical amino acids.

Methods for determining sequence identity include those described by Schuler, G. D. in Bioinformatics: A Practical Guide to the Analysis of Genes and Proteins, Baxevanis, A. D. and Ouelette, B. F. F., eds., John Wiley & Sons, Inc, 2001; Yona et al., in Bioiiformatics: Sequence, structure and databanks, Higgins, D. and Taylor, W. eds., Oxford University Press, 2000; and Bioinformatics: Sequence and Genome Analysis, Mount, D. W., ed., Cold Spring Harbor Laboratory Press, 2001. Methods to determine amino acid sequence identity are codified in publicly available computer programs such as GAP (Wisconsin Package Version 10.2, Genetics Computer Group (GCG), Madison, Wisc.), BLAST (Altschul et al., J. Mol. Biol. 215(3):403-10, 1990), and FASTA (Pearson, Methods in Enzymology 183:63-98, 1990, R. F. Doolittle, ed.).

In an embodiment of the present invention sequence identity between two polypeptides is determined using the GAP program (Wisconsin Package Version 10.2, Genetics Computer Group (GCG), Madison, Wisc.). GAP uses the alignment method of Needleman and Wunsch. (Needleman et al., J. Mol. Biol. 48:443453, 1970.) GAP considers all possible alignments and gap positions between two sequences and creates a global alignment that maximizes the number of matched residues and minimizes the number and size of gaps. A scoring matrix is used to assign values for symbol matches. In addition, a gap creation penalty and a gap extension penalty are required to limit the insertion of gaps into the alignment. Default program parameters for polypeptide comparisons using GAP are the BLOSUM62 (Henikoff et al., Proc. Natl. Acad. Sci. USA, 89:10915-10919, 1992) amino acid scoring matrix (MATrix=blosum62.cmp), a gap creation parameter (GAPweight=8) and a gap extension pararameter (LENgthweight=2).

Multi-Cistronic Configurations

Multi-cistronic replicons can be produced having different configurations. The different configurations can vary, for example, in the placement of a selection or reporter gene, the placement of non-structural genes, the placement and presence of structural regions, and the presence of more than two cistrons.

In an embodiment of the present invention, the GBV-B replicon is capable of replication in a cell, such as a human hepatoma cell, preferably a Huh7 cell; and comprises the following regions:
a GBV-B 5′ UTR substantially similar to bases 1445 of SEQ. ID. NO. 1;
a selection or reporter sequence functionally coupled to the GBV-B 5′ UTR;
an internal ribosome entry site;
a NS3-NS5B sequence substantially similar to bases 1938-7709 of SEQ ID NO: 1 functionally coupled to the internal ribosome entry site and a AUG translation initiation codon; and
a GBV-B 3′ UTR substantially similar to bases 7710-8069 of SEQ. ID. NO. 1.

Additional embodiments concerning the GBV-B replicon include one or more of following:
(1) The presence of a GBV-B structural region contiguous with the GBV-B 5′ UTR is present. The structural region may contain an additional region, such as NS2. Preferably, the GBV-B structural region comprises a sequence substantially similar to a sequence selected from the group consisting of: bases 446-511 of SEQ. ID. NO. 1, bases 446-487 of SEQ. ID. NO. 1, bases of 446-469 of SEQ. ID. NO. 1, the RNA version of bases 446-2641 (core-E2/p7) of SEQ. ID. NO. 2 and the RNA version of bases 446-3265 (core-NS2) of SEQ. ID. NO. 2;
(2) The presence of a GBV-B NS2 region or core region contiguous with the 5′ end of the NS3-NS5B region, preferably the NS2 region if present has a sequence substantially similar to the RNA version of bases 2642-3265 of SEQ. ID. NO. 2;
(3) The GBV-B 3′ UTR has a sequence substantially similar to bases 7710-8069 of SEQ. ID. NO. 1;
(4) The internal ribosome entry site has a sequence substantially similar to bases 1324-1934 of SEQ. ID. NO. 1; and
(5) The GBV-B replicon consists of the GBV-B 5′ UTR, a GBV-B structural region (which may include NS2), the selection or reporter sequence, the internal ribosome entry site, an NS3-NS5B or NS2-NS5B sequence, and a GBV-B 3′ UTR.

Reference to “the RNA version” indicates a ribose backbone and the presence of uracil instead of thymine.

Reference to a GBV-B region is not limited to a naturally occurring GBV-B region, but also includes derivatives of such regions. The scope of the derivatives is provided by a relationship (substantially similar) to a reference sequence.

Substantially similar nucleotide sequences have a nucleotide sequence identity of at least 85%, at least 95%, at least 99%, or 100%; and/or differ from each other by 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-25, 1-30, 1-35, 140, 1-45, or 1-50 nucleotides. Nucleotide differences between two sequences can be calculated by determining the minimum number of nucleotide modifications in which the two sequences differ. Nucleotide modifications can be deletions, additions, substitutions or any combination thereof. A preferred additional nucleotide sequence is a 5′ AUG sequence next to a coding sequence lacking a 5′ AUG.

Nucleotide sequence identity can be determined by methods well known in the art that compare the nucleotide sequence of one sequence to the nucleotide sequence of a second sequence and generate a sequence alignment. Sequence identity can be determined from the alignment by counting the number of aligned positions having identical nucleotides.

Methods for determining nucleotide sequence identity between two polynucleotides include those described by Schuler, in Bioinformatics: A Practical Guide to the Analysis of Genes and Proteins, Baxevanis, A. D. and Ouelette, B. F. F., eds., John Wiley & Sons, Inc, 2001; Yona et al., in Bioinformatics: Sequence, structure and databanks, Higgins, D. and Taylor, W. eds., Oxford University Press, 2000; and Bioinformatics: Sequence and Genome Analysis, Mount, D. W., ed., Cold Spring Harbor Laboratory Press, 2001. Methods to determine nucleotide sequence identity are codified in publicly available computer programs such as GAP (Wisconsin Package Version 10.2, Genetics Computer Group (GCG), Madison, Wisc.), BLAST (Altschul et al., J. Mol. Biol. 215(3):403-10, 1990), and FASTA (Pearson, W. R., Methods in Enzymology 183:63-98, 1990, R. F. Doolittle, ed.).

In an embodiment of the present invention, sequence identity between two polynucleotides is determined by application of GAP (Wisconsin Package Version 10.2, Genetics Computer Group (GCG), Madison, Wisc.). GAP uses the alignment method of Needleman and Wunsch. (Needleman et al., J. Mol. Biol. 48:443-453, 1970.) GAP considers all possible alignments and gap positions between two sequences and creates a global alignment that maximizes the number of matched residues and minimizes the number and size of gaps. A scoring matrix is used to assign values for symbol matches. In addition, a gap creation penalty and a gap extension penalty are required to limit the insertion of gaps into the alignment. Default program parameters for polynucleotide comparisons using GAP are the nwsgapdna.cmp scoring matrix (MATrix=nwsgapdna.cmp), a gap creation parameter (GAPweight-50) and a gap extension pararameter (LENgthweight=3).

Selection Sequence

A selection sequence in a GBV-B replicon can be used to facilitate the production of GBV-B replicon enhanced cells and replicon maintenance in a cell. Selection sequences are typically used in conjunction with some selective pressure that inhibits growth of cells not containing the selection sequence. Examples of selection sequences include sequences encoding antibiotic resistance and ribozymes.

Antibiotic resistance can be used in conjunction with an antibiotic to select for cells containing replicons. Examples of selection sequences providing for antibiotic resistance are sequences encoding resistance to neomycin, hygromycin, puromycin, or zeocin.

A ribozyme serving as a selection sequence can be used in conjunction with an inhibitory nucleic acid molecule that prevents cellular growth. The ribozyme recognizes and cleaves the inhibitory nucleic acid.

Reporter Sequence

A reporter sequence can be used to detect replicon replication or protein expression. Preferred reporter proteins are enzymatic proteins whose presence can be detected by measuring product produced by the protein. Examples of reporter proteins include, luciferase, beta-lactamase, secretory alkaline phosphatase, beta-glucuronidase, and green fluorescent protein and its derivatives. In addition, a reporter nucleic acid sequence can be used to provide a reference sequence that can be targeted by a complementary nucleic acid probe. Hybridization of the probe to its target can be determined using standard techniques.

Additional Sequence Configuration

Additional sequences are preferable 5′ or 3′ of a GBV-B genome or subgenomic genome region. However, the additional sequences can be located within a GBV-B genome as long as the sequences do not prevent detectable replicon activity. If desired, additional sequences can be separated from the replicon by using a ribozyme recognition sequence in conjunction with a ribozyme.

Additional sequences can be part of the same cistron as the GBV-B polyprotein or can be a separate cistron. If part of the same cistron, the additional sequences coding for a protein should result in a product that is either active as a chimeric protein or is cleaved inside a cell so it is separated from a GBV-B protein.

Selection and reporter sequences encoding a protein when present as a separate cistron should be associated with elements needed for translation. Such elements include a 5′ ribosome entry site.

Replicon Encoding Nucleotide Sequence

GBV-B replicons can be produced from a nucleic acid molecule coding for the replicon. A nucleic acid molecule can be single-stranded or part of a double strand, and can be RNA or DNA. Depending upon the structure of the nucleic acid molecule, the molecule may be used as a replicon or in the production of a replicon. For example, single-stranded RNA having the proper regions can be a replicon, while double-stranded DNA that includes the complement of a sequence coding for a replicon or replicon intermediate may useful in the production of the replicon or replicon intermediate.

Nucleic acid containing a sequence coding for a replicon can be produced from an expression vector. Replicons can be introduced into a cell as an RNA molecule in vitro transcribed from a corresponding DNA cloned in an expression vector, or can be isolated from a first cell expressing the expression vector and then transfected into a second cell.

An expression vector contains recombinant nucleic acid encoding a desired sequence along with regulatory elements for proper transcription and processing. The regulatory elements that may be present include those naturally associated with the nucleotide sequence encoding the desired sequence and exogenous regulatory elements not naturally associated with the nucleotide sequence. Examples of expression vectors are cloning vectors, modified cloning vectors, specifically designed plasmids and viruses.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. The translation of a particular codon into a particular amino acid is well known in the art (see, e.g., Lewin GENES IV, p. 119, Oxford University Press, 1990). Amino acids are encoded by codons as follows:
A=Ala-Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F-Phe-Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr-Threonine: codons ACA, ACC, ACG, ACU
V=Val=Valine: codons GUA, GUC, GUG, GUU
W=Trp=Tryptophan: codon UGG
Y=Tyr_Tyrosine: codons UAC, UAU.

### Detection Methods

Methods for detecting replicon activity include those measuring the production or activity of replicon RNA and encoded protein. Measuring includes qualitative and quantitative analysis.

Techniques suitable for measuring RNA production include those detecting the presence or activity of RNA. The presence of RNA can be detected using, for example, complementary hybridization probes or quantitative PCR. Techniques for measuring hybridization between complementary nucleic acid and quantitative PCR are well known in the art. (See for example, Ausubel, Current Protocols in Molecular Biology, John Wiley, 1987-1998, Sambrook et al., Molecular Cloning, A Laboratory Manual, 2^{nd} Edition, Cold Spring Harbor Laboratory Press, 1989, and U.S. Pat. No. 5,731,148.)

RNA enzymatic activity can be provided to the replicon by using a ribozyme sequence. Ribozyme activity can be measured using techniques detecting the ability of the ribozyme to cleave a target sequence.

Techniques for measuring protein production include those detecting the presence or activity of a produced protein. The presence of a particular protein can be determined by, for example, immunological techniques. Protein activity can be measured based on the activity of a GBV-B protein or a reporter protein sequence.

Techniques for measuring GBV-B protein activity vary depending upon the protein that is measured. Techniques for measuring the activity of different non-structural proteins such as NS3 and NSSB, are well known in the art. (See, for example, the references provided in the Background of the Invention.) Assays measuring replicon activity also include those detecting virion production from a replicon that produces a virion; and those detecting a cytopathic effect from a replicon producing proteins exerting such an effect. Cytopathic effects can be detected by assays suitable to measure cell viability.

Assays measuring replicon activity can be used to evaluate the ability of a compound to modulate GBV-B activities. Such assays can be carried out by providing one or more test compounds to a cell expressing a GBV-B replicon and measuring the effect of the compound on replicon activity. If a preparation containing more than one compound is found to modulate replicon activity, individual compounds or smaller groups of compounds can be tested to identify replicon active compounds.

Compounds identified as inhibiting GBV-B activity can be used to produce replicon enhanced cells and may be therapeutic or research compounds. The ability of a compound to serve as a therapeutic compound for HCV can be confirmed using animals susceptible to GBV-B and, if desired, through the subsequent use of a chimpanzee infected with HCV.

### Replicon Enhanced Cells

Replicon enhanced cells have an increased ability to maintain a replicon. Replicon enhanced cells can be produced by selecting for a cell able to maintain a GBV-B replicon and then curing the cell of the replicon.

Initial transfection can be performed using a replicon having a wild-type GBV-B sequence that contains at least a NS3-NS5B sequence or a functional derivative thereof. The replicon preferably contains a selection sequence to facilitate replicon maintenance.

Cells can be cured of replicons using different techniques such as those employing a replicon inhibitory agent. Replicon inhibitory agents inhibit replicon activity or select against a cell containing a replicon. Examples of such agents include IFN-α and compounds found to inhibit GBV-B replicon activity. The ability of a cured cell to be a replicon enhanced cell can be measured by introducing a replicon into the cell and determining efficiency of replicon maintenance and activity.

A first GBV-B replicon introduced into a replicon cured GBV-B cell may be the same or different than a second GBV-B replicon introduced into the GBV-B replicon enhanced cell. The two replicons can differ by the GBV-B coding sequences or by other sequences that may be present such as a selection sequence or a reporter sequence. Preferably, the first GBV-B replicon introduced into a GBV-B replicon enhanced cell has the same GBV-B sequences as the replicon that was used to produce the enhanced cell.

The overall method for producing a replicon enhanced cell can be summarized as involving the steps of: (a) introducing and maintaining a GBV-B replicon into a cell and (b) curing the cell of the replicon. In an embodiment of the present invention, the method further comprises: step (c) introducing and maintaining a GBV-B replicon into a cell, wherein the replicon may be the same or different from the step (a) replicon; and step (d) curing the cell of the replicon used in step (b), wherein the curing may be performed the same way or different from the technique employed in step (b). In a preferred embodiment for the production of an enhanced cell supporting a genomic replicon, step (a) is performed using a subgenomic replicon and step (c) is preformed using a genomic replicon.

### Virion Production

Genomic replicons can be used to produce virions. The produced virions have different uses such as providing for activities that can be measured and as a source of virus for infecting animals. Measuring virion activities under different conditions can be used to gain a better understanding of virion production and to assay the ability of a compound to alter such activities.

Preferably, genomic replicons are used to produce virions in replicon enhanced cells. Genomic replicons that can be used for virion production include those having a single cistron and those having multiple cistrons.

### GBV-B/HCV Chimerics

Chimeric GBV-B/HCV replicons infectious in a GBV-B susceptible animal provide for a further enhancement in using the animal as a surrogate model. The chimeric GBV-B/HCV replicon sequence would contain one or more HCV protein encoding regions or a portion thereof in a GBV-B scaffold. The GBV-B and HCV regions corresponding to 5′ UTR, core, E1, E2, NS2, NS3, NS4A, NS4B, NSSA, NS5B, and 3′UTR regions are well known in the art. (See, for example, references cited in the Background of the Invention and Hong et al., U.S. Publication Number U.S. 2001/0034019, Publication Date Oct. 25, 2001).

One or more GBV-B regions or a portion thereof present in a replicon described herein can be replaced with the corresponding region from HCV. In different embodiments the corresponding region encodes at least about 50 amino acids, at least about 75 amino acids, or an entire region present in a naturally occurring HCV.

Numerous examples of naturally occurring HCV isolates are well known in the art. HCV isolates can be classified into the following six major genotypes comprising one or more subtypes: HCV-1/(1a, 1b, 1c), HCV-2/(2a, 2b, 2c), HCV-3/(3a, 3b, 10a), HCV-4/(4a), HCV-5/(5a) and HCV-6/(6a, 6b, 7b, 8b, 9a, 11a). (Simmonds, J. Gen. Virol., 693-712, 2001.) Examples of particular HCV sequences such as HCV-BK, HCV-J, HCV-N, HCV-H, have been deposited in GenBank and described in various publications. (See, for example, Chamberlain et al., J. Gen. Virol., 1341-1347, 1997).

Replicon enhanced cells can be used to screen for chimeric GBV-B/HCV replicon sequences that can replicate and process viral polyprotein in a cell. The ability of functional chimeric GBV-B/HCV replicons to infect an animal such as a tamarin or owl monkey can then be evaluated.

### EXAMPLES

Examples are provided below to further illustrate different features of the present invention. The examples also illustrate useful methodology for practicing the invention. These examples do not limit the claimed invention.

### Example 1

### Techniques

This example illustrates techniques that can be employed for producing and analyzing GBV-B replicons and replicon enhanced cells.

Cell Lines and Culture Conditions

The human hepatoma cell line Huh7 was grown in high glucose Dulbecco's modified Eagle medium (DMEM; Life Technologies) supplemented with 2 mM L-glutamine, 100 U/ml of penicillin, 100 μg/ml streptomycin, 10% fetal bovine serum. Cells were subcultivated twice a week with a 1:5 split ratio. Aoutus trivirgatus (owl monkey) kidney cell line (OMK 637-69; ATCC number CRL-1556) was grown in minimum essential medium in Earle's BSS with non essential amino acids (MEM; Life Technologies) supplemented with 100 U/ml of penicillin, 100 μg/ml streptomycin, 10% fetal bovine serum. Saguinus oedipus (tamarin) lymphoblast cell line B95-8a, kindly provided by Dr. Fumio Kobune, was grown in RPMI 1640 medium (RPMI; Life Technologies) supplemented with 2 mM L-glutamine, 100 U/ml of penicillin, 100 μg/ml streptomycin, 10 mM HEPES, 1.0 mM sodium pyruvate, 10% fetal bovine serum. Neomycin-resistant lines were grown in the presence of G418 final concentration ranging between 0.250 and 1 mg/ml.

Plasmids Construction

GBV-B subgenomic replicon constructs were obtained by replacing the regions coding for structural proteins and NS2 protein with the sequences of neomycin phosphotransferase gene (neo) and EMCV internal ribosome entry site in the plasmid FL3/pACYC177 (EMBL accession number AJ277947). The FL3/pACYC177 plasmid encodes a GBV-B infectious full-length cDNA downstream of a T7 polymerase promoter. Neo and the EMCV internal ribosome entry site were joined stepwise to the GBV-B sequences by assembly-PCR and ligation reactions creating a unique AscI site at the junction between GBV-B 5′UTR and neo-gene.

The final GBV-B replicon sequence was moved as a BamHI-XhoI fragment into the more versatile pGBT9 vector (Clontech). Two SapI sites were removed from the neo-gene by primer-based mutagenesis leaving a SapI site at the 3′-end of the GBV-B coding sequence, useful for run-off transcription.

Four constructs, GBV-B-neo-RepA (neo-RepA), GBV-B-neo-RepB (neo-RepB), GBV-B-neo-RepC (neo-RepC) and GBV-B-neo-RepD (neo-RepD) were produced. Neo-Rep A contains the GBV-B 5′UTR followed by neo-gene. Neo-RepB, neo-repC and neo-repD contain the GBV-B 5′UTR, the ATG start codon and the subsequent 21, 39 and 63 nucleotides respectively of the GBV-B core coding sequence upstream of neo gene. The N-terminus of the neomycin phosphotransferase protein resulting from the described cloning design is preceded by three amino acids, depending on the addition of a cloning site upstream of neomycin phosphotransferase gene. Neo-RepB, neo-RepC and neo-RepD were obtained by replacing the original BamHI-AscI fragment of the neo-RepA clone with a BamHI-AscI fragment containing the ATG start codon and subsequent 21, 39 and 63 nucleotides of the GBV-B core coding sequence respectively. The RepD sequence is provided in FIG. 1.

Chimeric replicons bearing the HCV NS5B gene in place of the GBV-B corresponding gene were constructed in pGBT9 vector. Construction was achieved by replacing the SfiI-XhoI fragment of the GBV-B RepB clone, spanning NS5B region, with the corresponding fragment from a full-length chimeric clone containing NS5B of HCV, genotype 1a.

Bla-RepA, bla-RepB, bla-RepC, bla-RepD constructs were produced containing the β-lactamase gene (bla) in place of neo. The bla-Rep replicons was constructed by replacing in the GBV-B neo-Rep constructs the AscI-PmeI fragment spanning neo-gene with an AscI-PmeI fragment including the β-lactamase gene.

Mutants in the polymerase active site GDD motif were obtained by first constructing a GDD to GAA mutated neo-RepB clone by means of primer-based mutagenesis and subsequently replacing a restriction fragment spanning the mutation into the other wild type constructs.

RT-PCR amplification products of RNA from RepB76.1/Huh7 cells were subcloned in the pCR2.1 vector to perform sequencing.

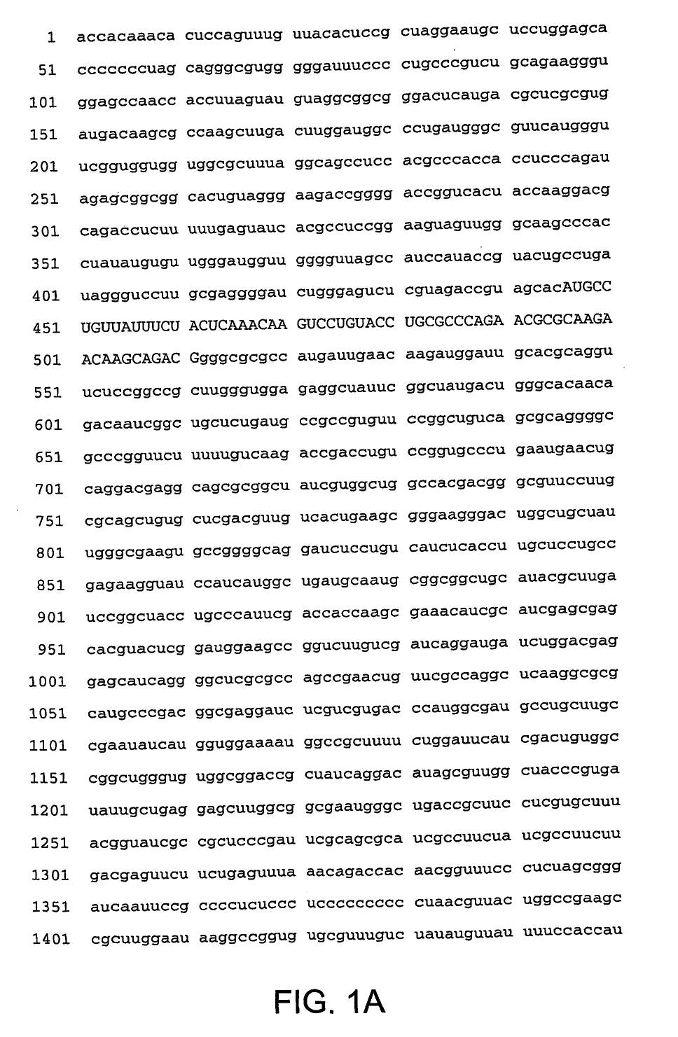

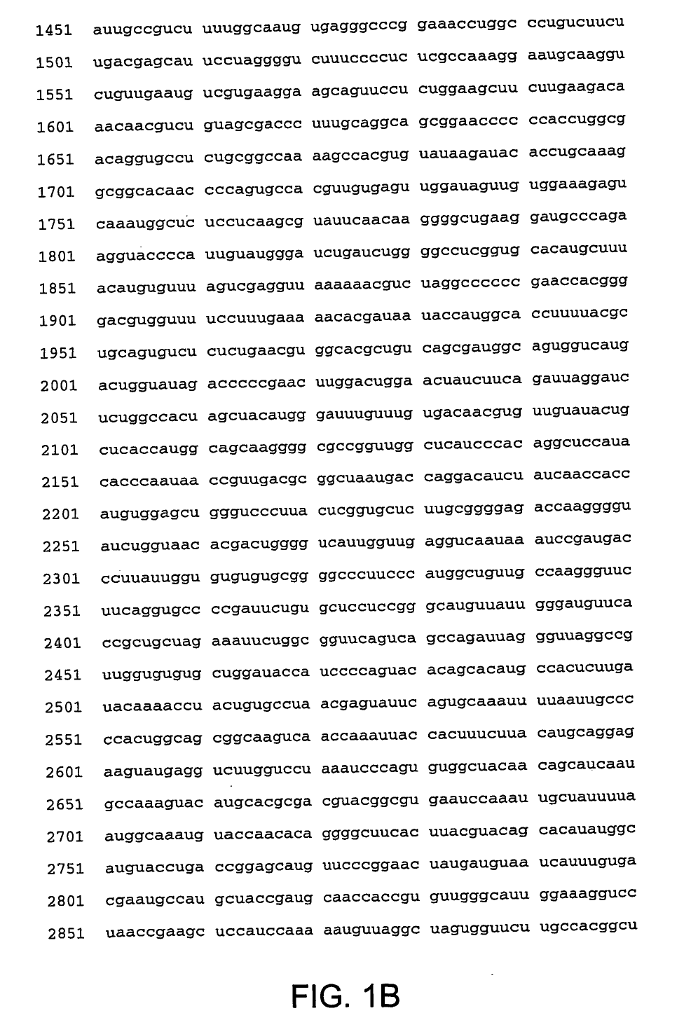

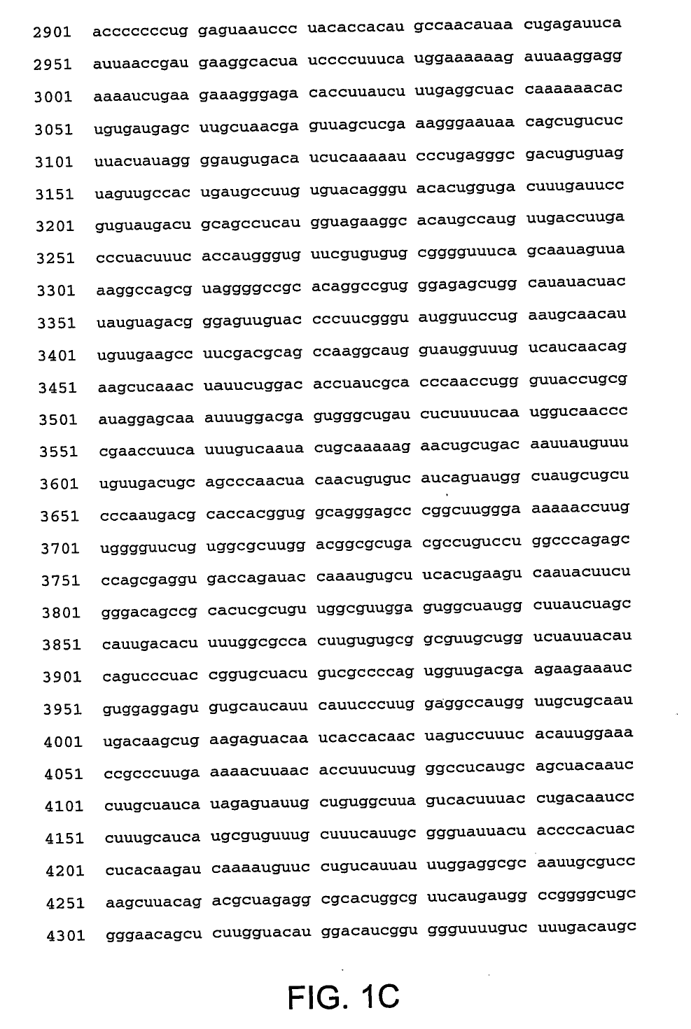

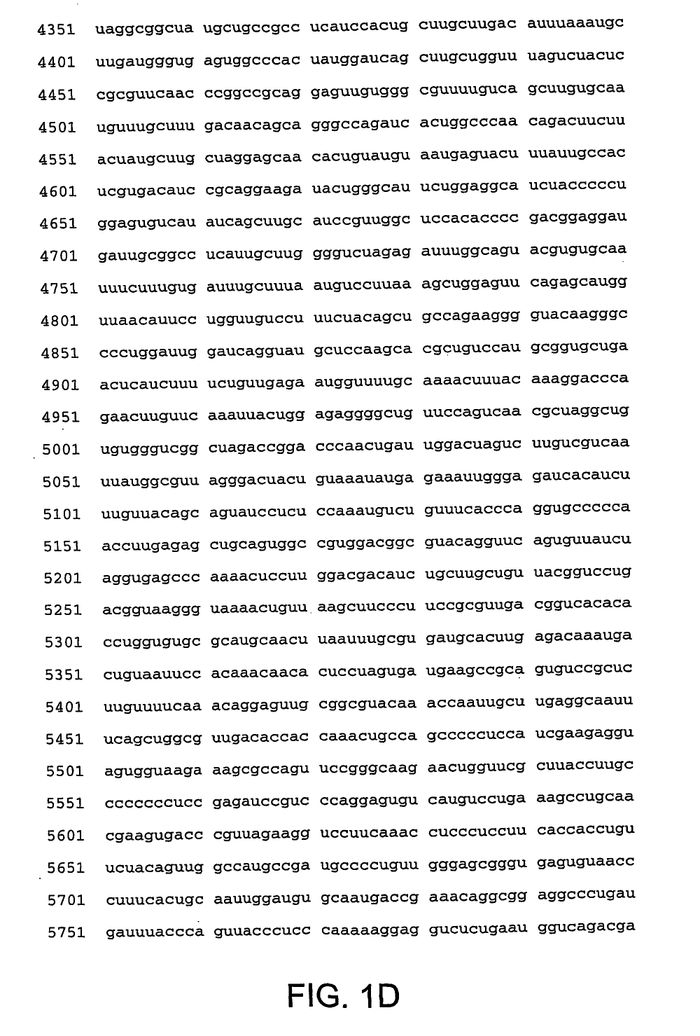

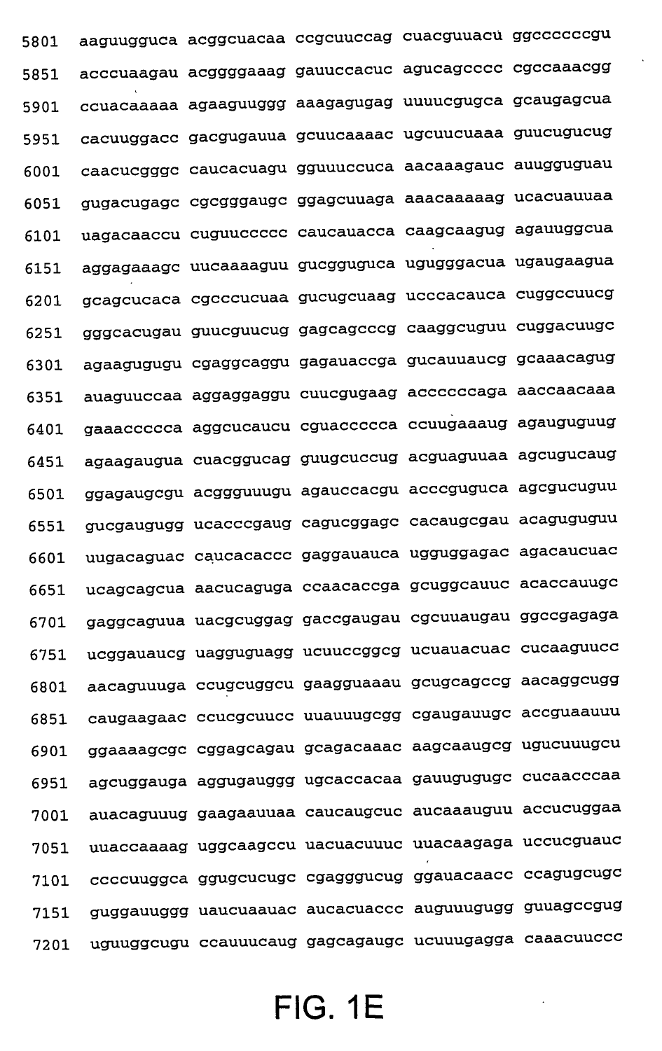

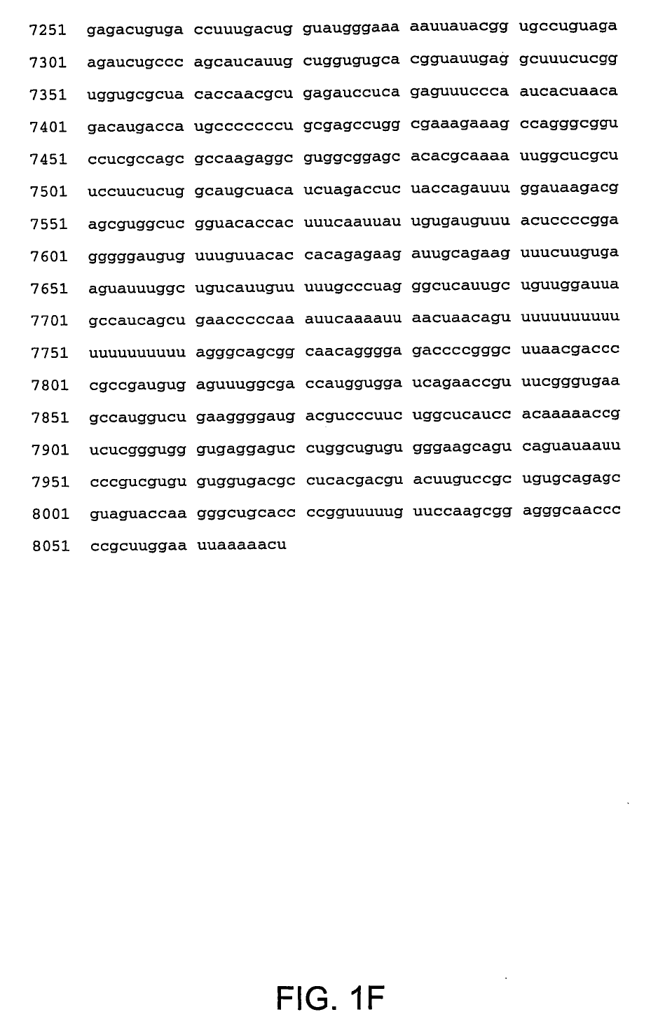

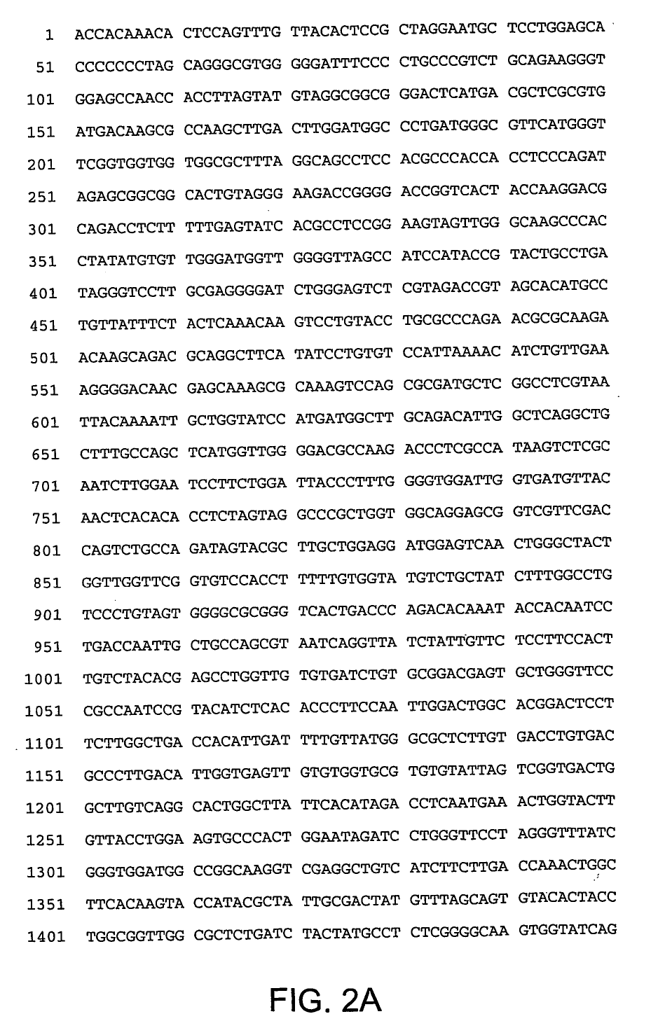

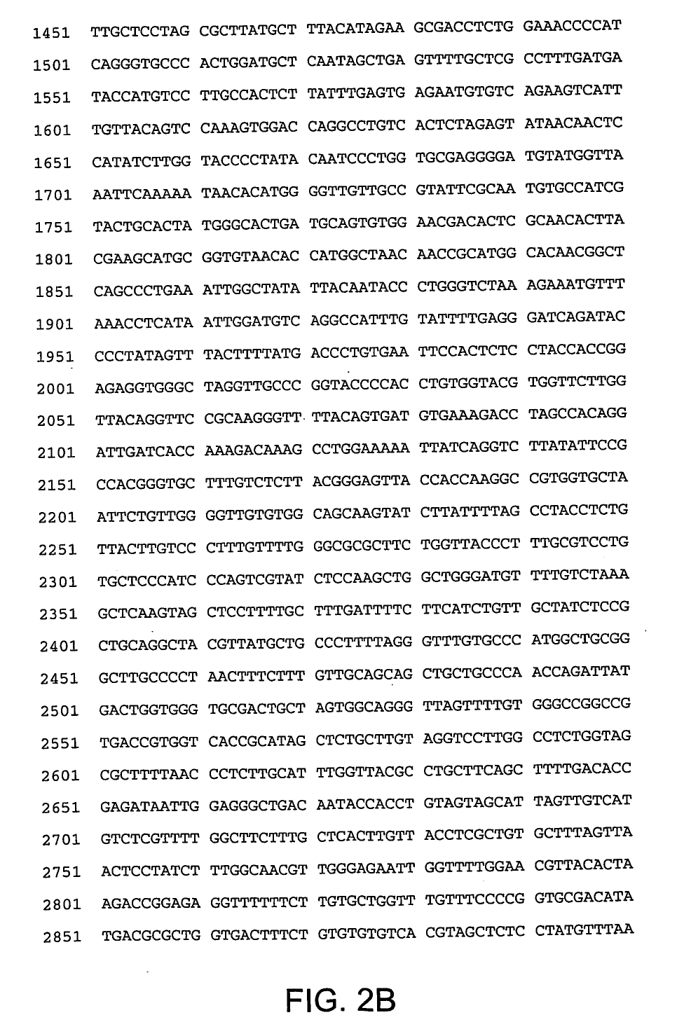

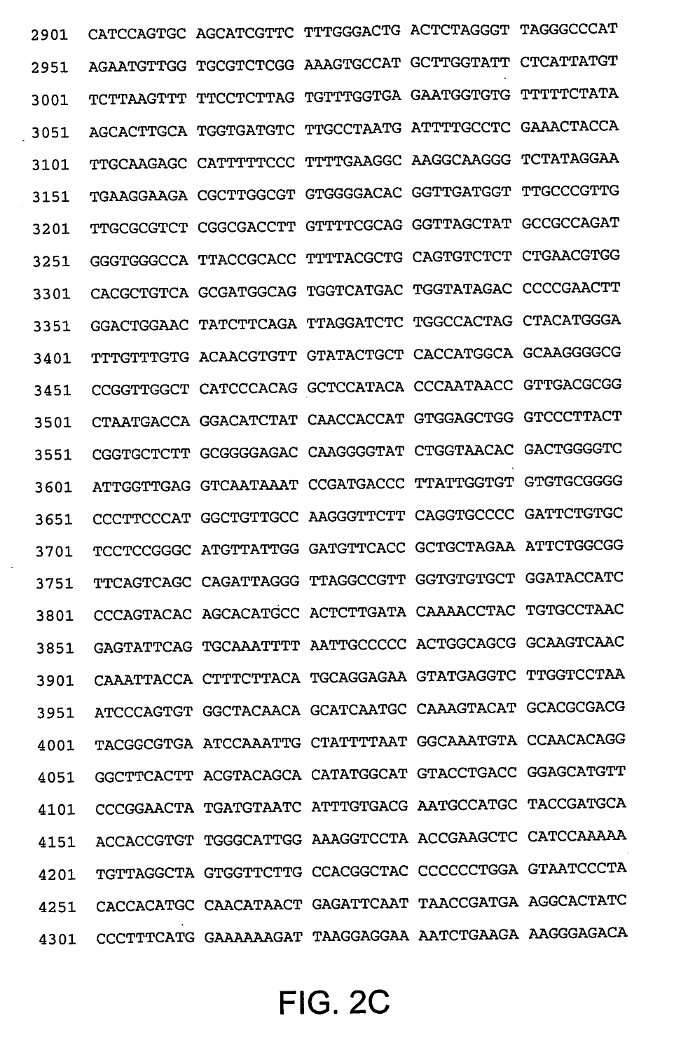

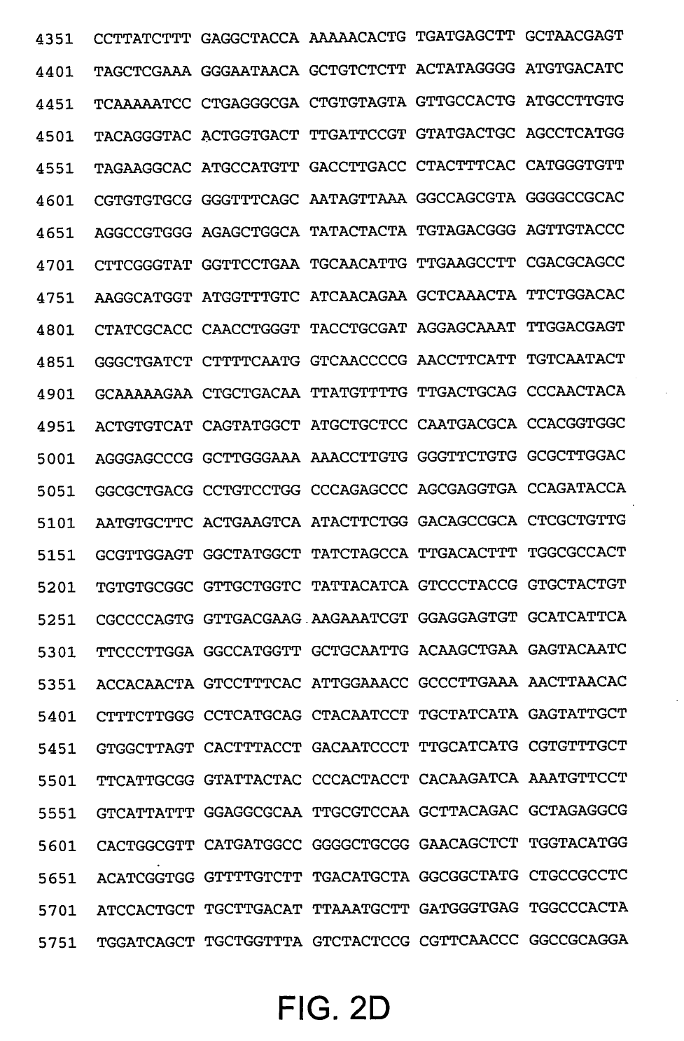

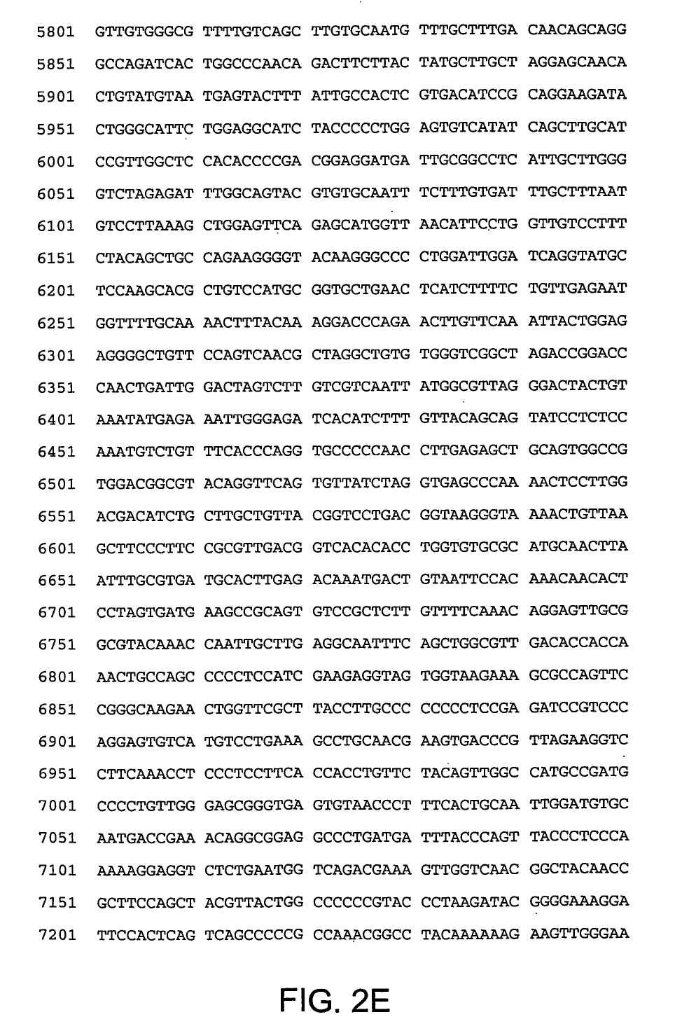

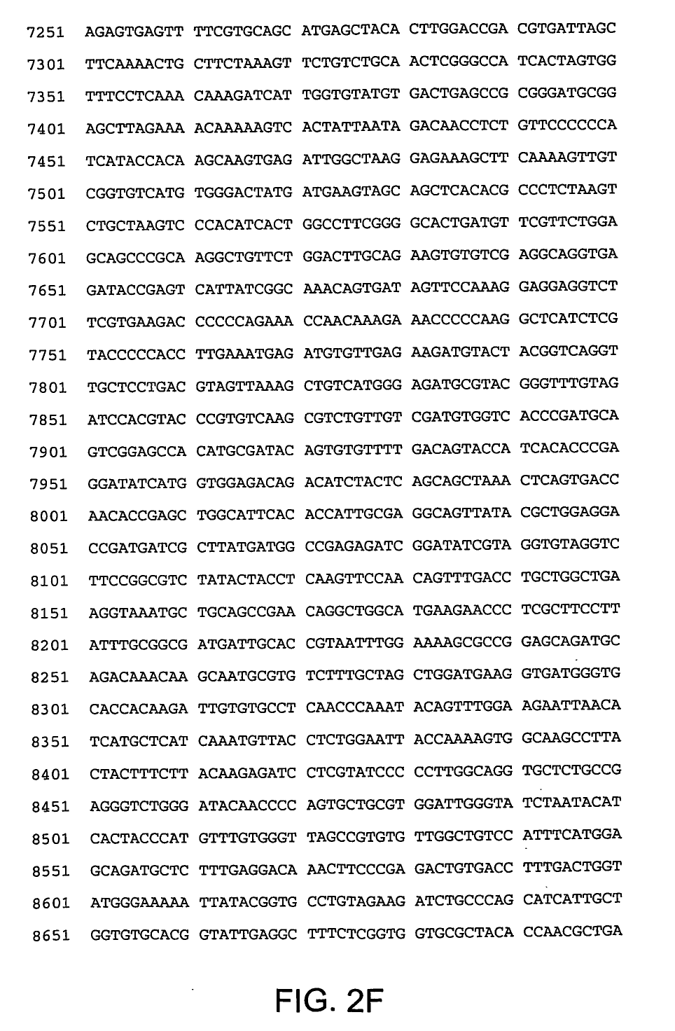

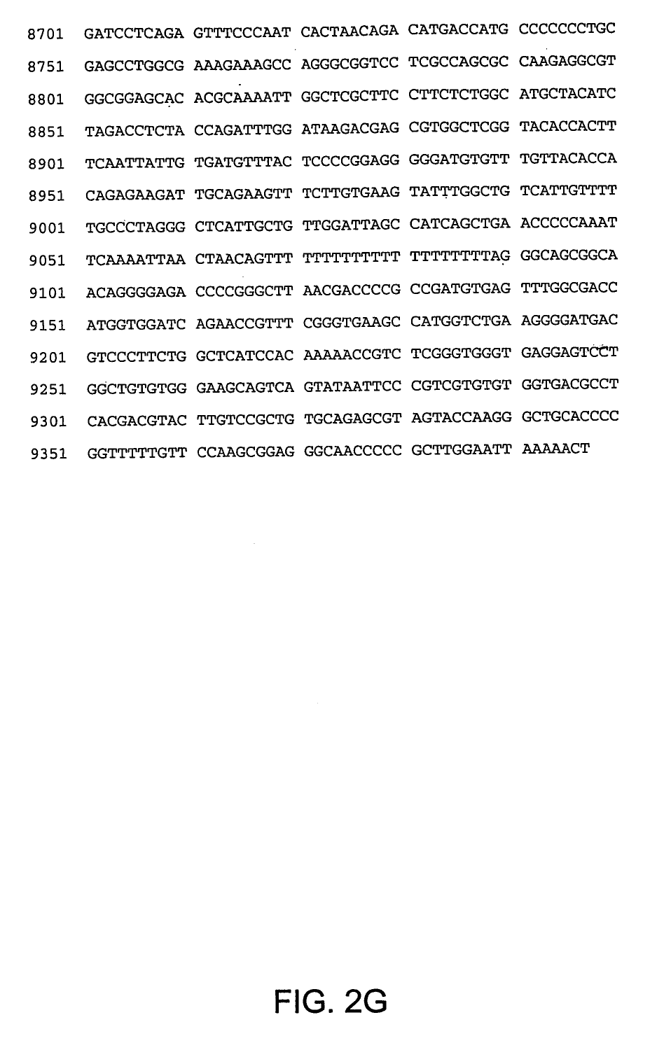

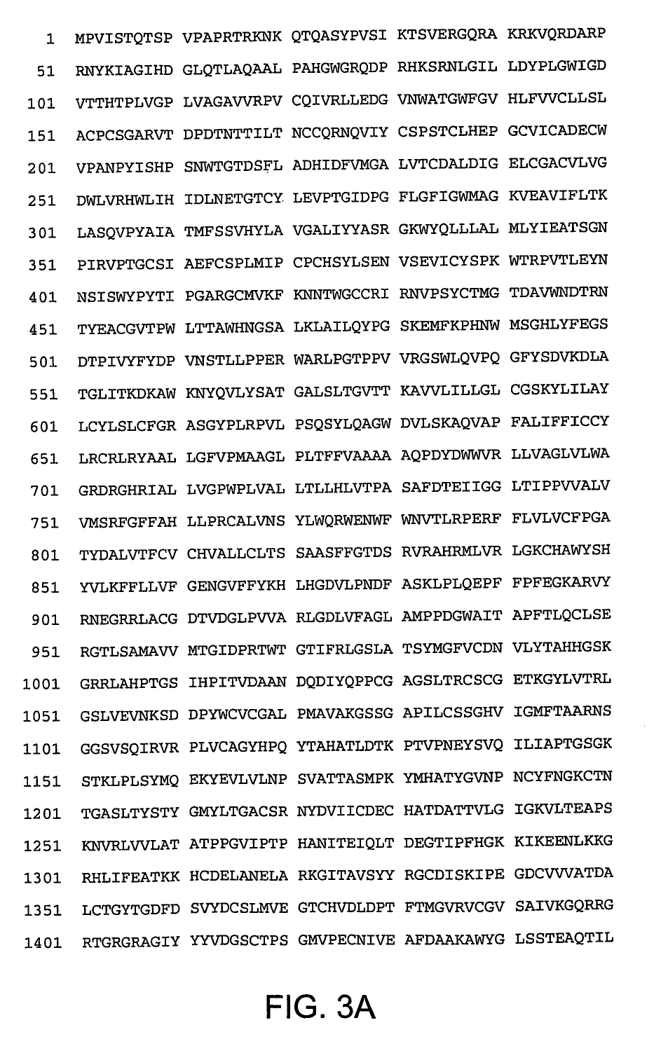

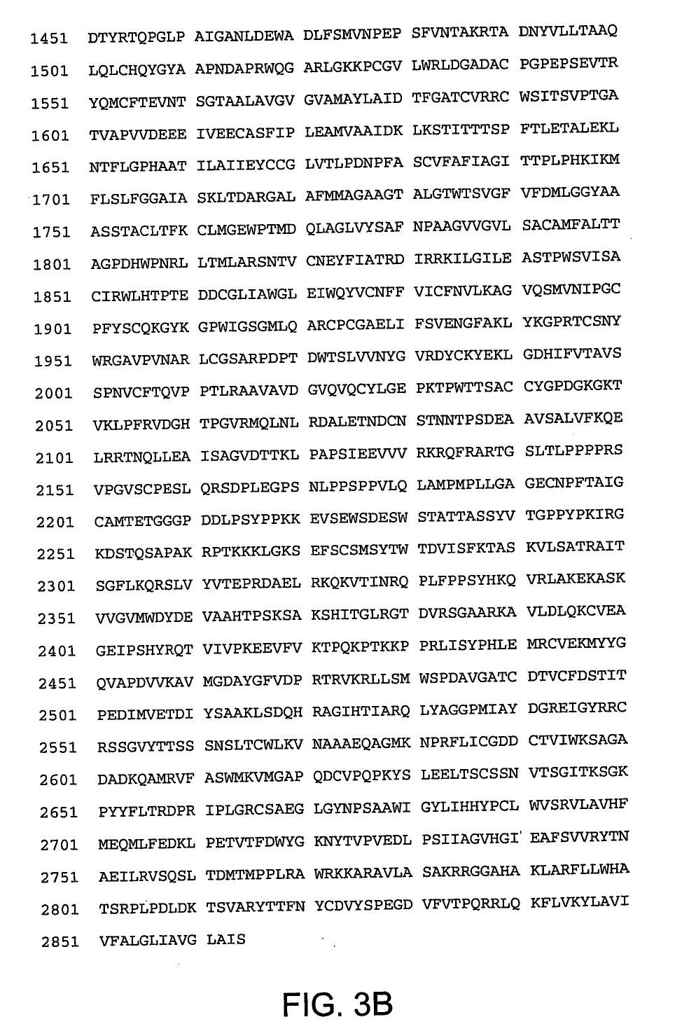

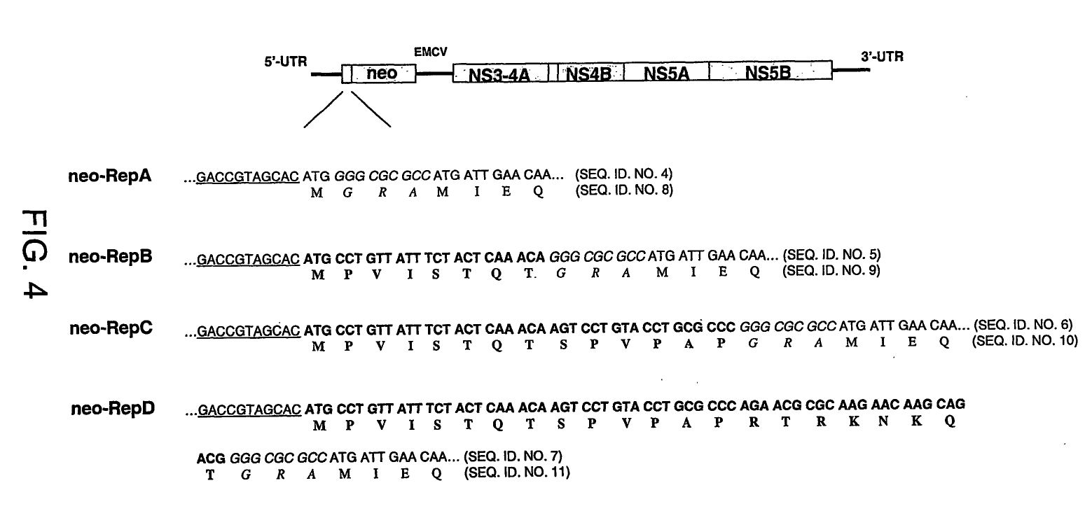

GBV-B genomic replicon constructs neo-FL-A, neo-FL-B, neo-FL-C and neo-FL-D (see FIG. 4 for neo-FL-D partial sequence, corresponding to neo-RepD partial sequence) were obtained by inserting the sequences of neomycin phosphotransferase gene (neo) and EMCV internal ribosome entry site within the plasmid FL3/pACYC177 (EMBL accession number AJ277947) upstream of the regions coding for the GBV-B structural proteins by means of routine molecular biology techniques. Neo-FL-A contains the GBV-B 5′UTR followed by neo-gene. Neo-FL-B, neo-FL-C and neo-FL-D contain the GBV-B 5′UTR, the ATG start codon and the subsequent 21, 39 and 63 nucleotides respectively of the GBV-B core coding sequence upstream of neo gene, as well as the corresponding neo-Rep subgenomic constructs. The N-terminus of the neomycin phosphotransferase protein resulting from the described cloning design is preceded by three amino acids, depending on the addition of a cloning site upstream of neomycin phosphotransferase gene.

Sequence Analysis

Sequencing was performed by the Big Dye Terminator Cycle sequencing kit with AmplyTaq (Applied Biosystems) and run with an Applied Biosystems model 373A sequencer.

In Vitro Transcription

SapI-linearized plasmids encoding G13V-B replicons were in vitro-transcribed by T7 RNA polymerase using an Ambion Megascript kit under nuclease-free conditions following the manufacturer's instructions. The reaction was terminated by incubation with DNAse I and precipitation with LiCl, according to the manufacturer's instructions. RNA was resuspended in nuclease free water, quantified by absorbance at 260 nm, immediately frozen in dry ice in 10 μg aliquots and stored at −80° C.

Electroporation Method and Monitoring of Replication

Human hepatoma Huh7 and derived cell lines, as well as monkey cell lines were used to test replication of GBV-B molecular constructs. Confluent cells from 15 cm diameter plate were divided 1:2. Cells were recovered after 24 hours in 5 ml medium, washed twice with 40 ml cold DEPC-treated PBS, filtered with Cell Striner filters (Falcon) and diluted in cold DEPC-treated PBS at a concentration of 10⁷ cells/ml. 2×10⁶ cell aliquots were subjected to electroporation with 10 μg of in vitro transcribed RNA by 2 pulses at 0.35 KV and 10 μF using a BioRad Genepulser II.

Immediately after electric pulses cells were diluted in 8 ml complete Dulbecco's Modified Eagle Medium (DMEM) and processed with different protocols depending on the selection/tracer used. In the case of neo-constructs transformation, cells were divided in 3 plates of 15 cm diameter and on the following day the selecting antibiotic G418 (Sigma G-9516) was added at a concentration of 0.250, 0.5 or 1 mg/ml. In 2 weeks neomycin-sensitive cells died and at the fourth week surviving cell clones were observed for the 0.25 mg/ml concentration. Surviving clones were picked-up and expanded by growing them in individual plates. When a bla reporter gene was used, 1.5 ml, 1.0 ml and 0.5 ml of transfected cells suspension were plated in each well of Multiwell-6-wells plate (Falcon cat. N. 35-3046) to be stained respectively at 24, 48 and 72 hours.

Aurora substrate system “CCF4” was used to measure β-lactamase activity. When quantitative PCR was used to measure transient replication, cells were plated 1-2×10⁵/well in “6-multi-well plate”. After 3 days total RNA was purified as described in the TRIzol protocol (Life Technologies) and 10 out of 100 μl of total RNA were used in individual TaqMan reactions.

TaqMan Quantification of GBV-B RNA

GBV-B RNA was quantified by a real-time 5′ exonuclease PCR (TaqMan) assay using a primer/probe set that recognized a portion of the GBV-B 5′UTR. The primers (GBV-B-F3, GTAGGCGGCGGGACTCAT (SEQ. ID. NO. 14), and GBV-B-R3, TCAGGGCCATCCAAGTCAA (SEQ. ID NO. 15)) and probe (GBV-B-P3,6-carboxyfluorescein-TCGCGTGATGACAAGCGCCAAG (SEQ. ID. NO. 16)-N,N,N′,N′-tetramethyl-6-carboxyrhodamine) were selected using the Primer Express software (PE Applied Biosystems). The fluorescent probe was obtained from PE Applied Biosystems. The primers were used at 10 pmol/50 μl reaction, and the probe was used at 5 pmol/50 μl reaction.

PCR was were performed using a TaqMan Gold RT-PCR kit (PE Applied Biosystems). PCR included a 30 minute reverse transcription step at 48° C., followed by 10 minutes at 95° C. and by 40 cycles of amplification using the universal TaqMan standardized conditions (a 15 second at 95° C. denaturation step followed by a 1 minute 60° C. annealing/extension step).

RNA transcribed from a plasmid containing the first 2000 nucleotides of the GBV-B genome was used as a standard to establish genome equivalents. Standard RNA was transcribed using a 17 Megascript kit (Ambion) and was purified by DNase treatment, phenol-chloroform extraction, Sephadex-G50 filtration and ethanol precipitation. RNA was quantified by absorbance at 260 nm and stored at −80° C.

All reactions were run in duplicate by using the ABI Prism 7700 Sequence Detection System (PE Applied Biosystems). A primer set for human GAPDH mRNA (PE Applied Biosystems) was used as an endogenous reference. Transfected RNA obtained by in vitro transcription of mutant constructs in which the sequence coding for the GDD motif in the active site of NS5B polymerase was replaced by GAA was used as calibrator. Results from two independent experiments were analyzed using both the Comparative Ct Method and the standard curve method.

Preparation of Proteins, Genoinic DNA and Total RNA

Total RNA, genomic DNA and total proteins were purified from cells grown in monolayer with TRIzol reagent (Life Technologies) following the manufacturer's instructions.

Northern Blot

8 μg of total RNA extracted from Huh7 cells and Huh7-derived GBV-B replicon cell clones was subjected to electrophoresis on a 1% agarose/formaldehyde gel, blotted onto Amersham's Hybond-N⁺ membranes and hybridized to a GBV-B RNA probe. Electrophoresis, blot and hybridization procedures were performed following the protocols of Amersham's Hybond-N⁺ membranes instruction manual with slight modifications. The [α³²P]-CTP-labelled RNA probe was produced by in vitro transcription of a GBV-B genome fragment (nt 4641-6060 of the FL3 genome sequence) cloned in pCR2.1 vector under the T7 promoter in the orientation producing a negative stranded transcript.

Non-Quantitative RT-PCR

Total RNA was used for first strand cDNA synthesis by Superscript II reverse transcriptase (Gibco-BRL) under the manufacture's conditions. PCR amplification was performed using Elongase enzyme mix (Gibco-BRL) or Taq DNA polymerase (Promega). Primers were purchased from MWG (Germany).

Test of Putative Inhibitors of GBV-B Replication

Huh7 cell clones carrying GBV-B or HCV replicons were used to test the effect of human interferon alpha-2b. Cells (1×10⁵) were plated into each well of a series of wells of Multiwell-6-wells plates (falcon cat N. 35-3046) in medium without G418. After 16 hours the medium was discarded and increasing concentrations of the test compound in fresh medium were added to each series of wells. Controls were run using the specific compound solvent at the appropriate dilution.

Cells were grown up to 3 days in the presence of compounds or compound solvent without a compound, avoiding cell confluence, and finally lysed with TRIzol. Total RNA was purified as described in the TRizol protocol (Life Technologies). Ten out of 100 μl of total RNA were used in each reaction.

Taq Man analysis was performed using a neomycin primer set (taq NEO 1, GATGGATTGCACGCAGGTT (SEQ. ID. NO. 17), and taq NEO 5, CCCAGTCATAGCCGAATAGCC (SEQ. ID. NO. 18)) and a NEO probe (1,6-carboxyfluorescein-TCCGGCCGCTTGGGT GGAG (SEQ. ID. NO. 19)-N,N,N′,N′-tetramethyl-6-carboxyrhodamine). Human GAPDH mRNA quantified with a specific primer set (PE Applied Biosystems) was used as an endogenous reference. GBV-B RNA extracted from mock-treated cells was used as a calibrator. Results from two independent experiments were analyzed using both the Comparative Ct Method and the standard curve method.

Western Blot

Protein extracts were prepared from 1×10⁶ cells by TRIzol extraction and fractionated on a 10% SDS-PAGE (30-100 μg/slot). The gel was blotted onto a nitrocellulose filter by routine methodologies. The filter was incubated with a 1:50 dilution of a pool of four tamarin sera previously tested as immunoreactive against GBV-B antigens (Sbardellati et al., J. Gen. Virol. 82:2437-2448, 2001) in blocking buffer (5% non-fat dry milk, 0.05% Tween-20 in TBS). The filter was washed 5 times with blocking buffer and was then incubated with a mouse anti-monkey antibody (Sigma). After 5 more washes the filter was incubated with a HRP-conjugated mouse antibody and finally treated with West Pico Supersignal chemiluminescent substrate (PIERCE) following manufacturer's instructions and the signals detected by X-ray film exposure.

### Example 2

### Cloning GBV-B Neo-Resistant Replicons and Transfection of Mammalian Cells

The FL-3 plasmid was used as a parental molecule to build-up GBV-B subgenomic replicon constructs. The FL-3 plasmid encodes a tamarin infectious GBV-B genomic sequence. (Sbardellati et al., J. Gen. Virol. 82:2437-2448, 2001).

GBV-B bicistronic replicons were designed as schematized in FIG. 4. In the first cistron the GBV-B 5′UTR sequence directs translation of the neomycin phosphotransferase selectable marker; the second cistron is formed by the EMCV internal ribosome entry site directing translation of the GBV-B non-structural proteins from NS3 to NS5B; downstream of the coding region is the complete GBV-B 3′UTR sequence.

Four replicon versions varying in the 5′UTR/neo-gene boundary are illustrated in FIG. 4: neo-RepA, neo-RepB, neo-RepC and neo-RepD. In neo-RepA the GBV-B internal ribosome entry site containing 5′UTR was inserted up to the ATG polyprotein starting codon, thus directing the translation of neomycin phosphotransferase with no upstream GBV-B core coding sequence. In neo-RepB a stretch of 21 nucleotides coding for the first 7 amino acids of core protein following ATG was included producing a neo-gene N-terminal fusion protein. In neo-RepC a stretch of 39 nucleotides coding for the first 13 amino acids of core protein following ATG was included producing a neo-gene N-terminal fusion protein. In neo-RepD a stretch of 63 nucleotides coding for the first 21 amino acids of core protein following ATG was included producing a neo-gene N-terminal fusion protein. The N-terminus of the neomycin phosphotransferase protein resulting from the described cloning design in all the neo-Rep constructs was preceded by three more amino acids (GRA) depending on the addition of the cloning site upstream of neomycin phosphotransferase gene.

Neo-Rep plasmids were in vitro transcribed after linearization at an engineered SapI sites to generate GBV-B subgenomic transcripts terminating at the precise 3′-end of the genomic infectious molecules. In vitro transcribed RNA was transfected into Huh7 human hepatoma cells by electroporation. RNA from neo-RepB-GAA plasmid, mutated in the active site of the NS5B polymerase, was used as a negative control.

After 24 hours of growth in the absence of selection, neomycin (G418) was added to the medium at 0.250 mg/ml. After 30 days of culture in the presence of G418, resistant clones were picked-up and grown as individual cell lines.

In a typical experiment, 64 neo-resistant colonies upon transfection of 2×10⁶ cells with 10 μg of neo-RepA RNA were selected, 793 colonies upon transfection of neo-RepB were selected, 780 colonies upon transfection with neo-RepC were selected, and 1920 colonies upon transfection with neo-RepD were selected. Parallel attempts to isolate neo-resistant clones upon selection with higher G418 concentrations failed. However, the concentration of G418 could be increased for at least some of the cell clones, once the clones were isolated and individually grown.

The neo-resistant individual cell lines showed some variability in the growth rate. A fast-growing clone kept at 1 mg/ml G418 was designated “B76.1/Huh7”.

B76.1/Huh7 was deposited in accordance with the Budapest Treaty at the Advanced Biotechnology Center (ABC), Interlab Cell Line Collection, (Biotechnology Dept.), Largo Rossana Benzi, 10, 16132 Genova, Italy. The deposited B76.1/Huh7 was assigned deposit number PD02002 and a deposit date of 22 Jan. 2002.

Attempts to reproduce successful transfection of GBV-B subgenomic replication using as recipients primate (tamarin and owl monkey) cell lines of non-hepatic origin failed. Additionally, RNA transcribed from the chimeric HCVpol/GBV-B replicon bearing the HCV NS5B gene in place of GBV-B counterpart was unable to replicate in cells tested.

### Example 3

### Detection and Quantification of GBV-B RNA in Transfected Huh7 Cells

RNA was extracted from several individual neo-RepB/Huh7 cell clones and subjected to non-quantitative RT-PCR using various sets of primers. PCR products was obtained only when a reverse transcription step was included, indicating that amplification was exclusively RNA-dependent and not due to the presence of residual DNA in the RNA preparation. Moreover, the integration of replicon copies in host genomic DNA was also excluded by lack of amplification of both GBV-B and neo-gene sequences from cell clones genomic DNA preparations.

To obtain quantitative measurements of replicon RNA molecules in the neo-resistant clones, Taqman RT-PCR using primers and a probe complementary to GBV-B 5′UTR region was performed on individual cell clones selected upon transfection of neo-RepB RNA. The results, summarized in Table 1, confirmed the RNA-dependent amplification of replicon sequences and showed that the number of GBV-B genome equivalents (G.E.)/cell was variable, ranging between 30 and 100. The raise in G418 concentration resulted in a 3-fold increase in G.E./cell, as shown in Table 1 for clone B76.1/Huh7.

TABLE 1
Comparison of neo-RepB copy numbers in individual
neo-resistant cell lines.

| Cell line | G.E./μg cell RNA | mean G.E./cell |
|---|---|---|
| B4 | 3.24 × 10⁶ | 32.4 |
| B57 | 3.43 × 10⁶ | 103.0 |
| B59 | 2.68 × 10⁶ | 80.5 |
| B76 | 1.40 × 10⁶ | 35.0 |
| B76.1* | 4.85 × 10⁶ | 121.0 |
| B78 | 1.30 × 10⁶ | 38.9 |
| B86 | 0.58 × 10⁶ | 29.3 |

Cell lines were grown at 0.250 mg/ml G418;
*cells grown at 1 mg/ml G418.
The amount of total cellular RNA was measured determining absorbance at 260 nm.

### Example 4

### Detection of GBV-B Proteins

GBV-B NS3 protein produced from replicon clones was visualized by Western blot experiments performed with extracts of individual cell clones. A pool of GBV-B-infected tamarin sera, in which seroconversion had already been detected (Sbardellati et al., J. Gen. Virol. 82:2437-2448, 2001), was used as an immunological reagent to GBV-B proteins. The results show a specific band at the expected molecular weight for NS3 that is not present in the mock-transfected cells. The identification of NS3 protein was confirmed using a purified GBV-B NS3 preparation as a positive control.

The reactivity of those sera to NS5B, which has a size similar to NS3, though detectable by ELISA by coating a purified antigen (Sbardellati et al., J. Gen. Virol. 82:2437-2448, 2001), was not detectable by Western blot. This was confirmed by the lack of signal to purified NS5B used as a positive control.

### Example 5

### Effect of Antiviral Compounds on GBV-B RepB/Huh7 Clones

The effect of human alpha-interferon (α-IFN) and other chemical compounds on the GBV-B replicon system was determined to evaluate the susceptibility of the GBV-B replicon to HCV antiviral agents. The effect of different antiviral agents were determined using B76.1/Huh7 cells alone or in parallel with 10A cells.

Experiments were performed using 10⁵ cells of B76.1/Huh7 and 10A plated into individual wells of 6-multiwell plate and incubated overnight in the absence of G418. The next day, the medium was replaced with fresh medium containing serial dilutions of test compound. Cells were grown up to 3 days in the presence of the test compound or the compound solvent, taking care that confluence was not reached to avoid a specific growth inhibition. (Pietschmann et al., J. Virol. 75:1252-1264, 2001.) RNA was extracted and TaqMan analysis was performed using a primers-probe set specific for the neomycin gene in order to avoid any methodological difference in the measurement of the HCV and GBV-B RNA molecules.

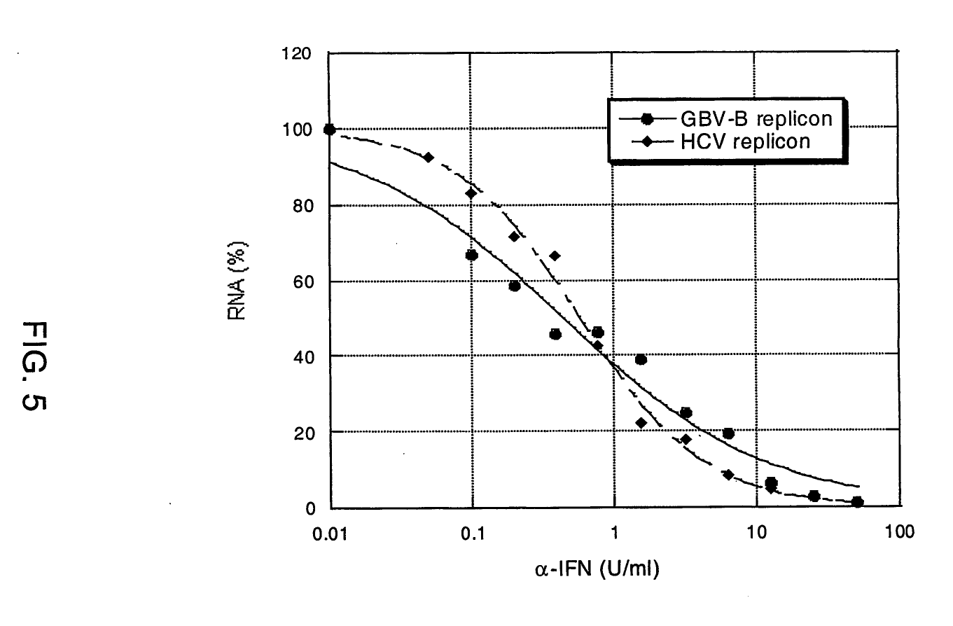

The effects of human alpha-IFN is shown in FIG. 5. Human alpha-IFN is approved for treating hepatitis C infection and reportedly acts on HCV replicon. (Frese et al., J. Gen. Virol. 82:723-733, 2001, Guo et al., J. Virol. 75:8516-8523, 2001.) Human alpha-IFN has a comparable effect on GBV-B and HCV replicons with an IC50 of 0.45 U/ml for GBV-B and of 0.58 for HCV.

### Example 6

### GBV-B Replicon Sequence Variation

GBV-B replicon RNA molecules able to replicate in Huh7 showed no sequence variation with respect to the parental full-length infectious RNA. Portions of GBV-B replicon spanning the complete replicon were amplified by RT-PCR of total RNA of B76.1/Huh7 cells and subcloned for sequencing. Two subclones per each region obtained from independent RT-PCRs were sequenced.

No mutation was consistently found in both individual subclones analyzed per region suggesting the absence of adaptive mutations. Sporadic mutations present only in one of the two subclones of each GBV-B region were observed. The sporadic mutations were attributed to PCR errors. An alternative explanation it that a mixed replicon RNA population exists in this cell line in which no mutation is present in every molecule.

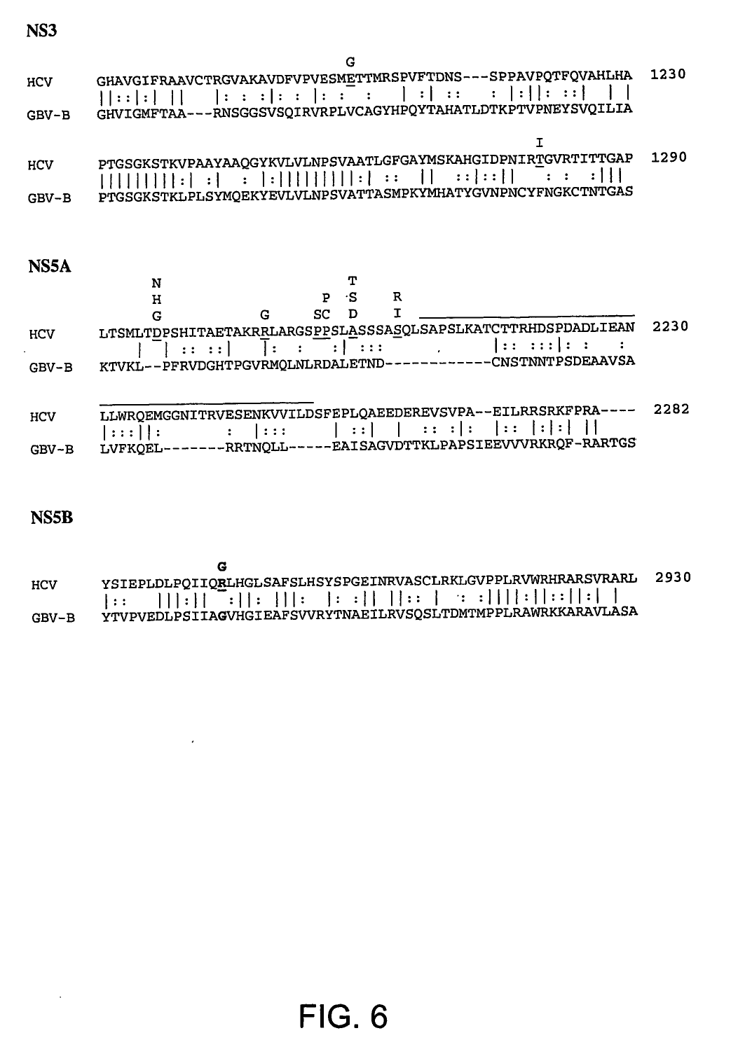

The position of adaptive mutations reported for an HCV replicon (Bartenschlager et al., Antiviral Res. 52:1-17, 2001) was compared with the corresponding amino acid residue in the sequence deduced for the GBV-B replicon. Results are reported in FIG. 6 and show the presence in GBV-B of “HCV adapted” amino acids in only one case, the HCV mutation R2884G in NS5B (Lohmann et al., J. Virol. 75:1437-1449, 2001), being a glycine residue present in GBV-B replicating RNA.

### Example 7

### Replicon Enhanced Cells

Replicon enhanced cells had an increased transfection and replication efficiency for GBV-B replicons. The effect of replicon enhanced cells was evaluated by eliminating the replicon RNA from B76.1/Huh7 cells and comparing the cured cells cB76.1/Huh7 with parental Huh7.

A B76.1/Huh7 cell culture was cured of replicon RNA using a high concentration of human α-IFN for a time sufficient to achieve total inhibition of replication and complete degradation of the resident GBV-B replicon RNA molecules. B76.1/Huh7 was maintained in culture with 100 U/ml α-IFN for 15 days in the absence of neomycin. The disappearance of the selectable RNA replicon molecules was checked at the end of the treatment by TaqMan analysis and confirmed by the inability of the “cured” clone to grow in the presence of the selector neomycin.

The resulting cured cell line was designated “cB76.1/Huh7”. cB76.1/Huh7 was transfected with in vitro transcribed RNA from the neo-RepB plasmid, and kept under neomycin selection. More than 80% of the cells used for transfection survived, indicating an increase of productive transfection of thousand folds respect to wild type Huh7 cells.

cB76.1/Huh7 was deposited in accordance with the Budapest Treaty at the Advanced Biotechnology Center (ABC), Interlab Cell Line Collection, (Biotechnology Dept.), Largo Rossana Benzi, 10, 16132 Genova, Italy. The deposited cB76.1/Huh7 was assigned deposit number PD02001 and a deposit date of 22 Jan. 2002.

The increased efficiency of replication of RepB RNA in cB76.1/Huh7 cells compared to wild type Huh7 cells was also monitored using a colorimetric assay after transfection of RNA transcribed from bla-RepB plasmid. The bla-RepB plasmid expresses a α-lactamase marker in place of the selector neomycin phosphotransferase. The results with α-lactamase-depending system were in overall agreement with the data obtained exploiting neomycin resistance, taking into account the different sensitivity of the systems. Treatment of bla-RepB transfected cB76.1/Huh7 cells with α-IFN reduced the number of blue cells to background levels, demonstrating that the stain was actually depending on RepB replication.

Quantitative analysis of transient replication was performed by real-time TaqMan. Three days after transfection a 20-30 fold increase of RepB RNA with respect to a non-replicating control in cB76.1/Huh7 cells was observed, whereas RepB RNA was below the detection limits in unselected Huh7. The data indicate that the cell giving rise to the clone B76.11Huh7 was capable of sustaining replication at a higher extent than the majority of the other cells in the originally transfected Huh7 population.

Eight independent cell lines originally transfected with neo-RepB RNA and kept for 2 weeks in the absence of G418 were transfected with RNA transcribed from the bla-RepB plasmid. All cell lines supported replication with an efficiency much higher than that shown by the non-clonal Huh7 original population (0.005% true blue cells, 3% pale blue cells), ranging from 10 to 80% blue cells, and with a certain degree of variability in the stain intensity among the various clones. This indicates that most of the originally identified neo-resistant clones were actually originated from the selection of transfected cells in the total Huh7 population able to enhance replication of GBV-B replicon RNA with respect to a base-line lying below the detection threshold of the selection system used.

### Example 8

### Use of Enhanced Cells to Achieve Replication of Full-Length GBV-B

GBV-B full-length genomic FL3 RNA (EMBL accession number AJ277947) was transfected in Huh7 and in enhanced cells cB76.1/Huh7 in parallel with the corresponding GAA control. Intracellular GBV-B RNA was measured by Taqman in a time-course experiment.

Replicon enhanced cells were able to support full length genomic replication. In contrast, transfection of Huh7 with FL3 failed to provide detectable FL3 replication.

Results of transfection of cB76.1/Huh7 enhanced cells showed that after 6 days the amount of FL3 RNA extracted from about 5×10⁵ cells was 1.15×10⁶ G.E., whereas the RNA extracted from the same number of cells transfected with the mutant GAA construct corresponded to 1.15×10⁵ G.E. Treatment of the transfected cells with interferon resulted in a 10-20-fold decrease of FL3 RNA amount and did not affect the GAA mutant RNA amount. The lack of sensitivity to interferon of the GAA mutant indicated that the GAA RNA detectable at day 6 post transfection corresponded to non-replicated input RNA.

### Example 9

### Infection of Enhanced Cells with GBV-B

GBV-B virus inoculum produced upon passage of virus in tamarins was used to infect Huh7 and cB76.1/Huh7 enhanced cells. GBV-B containing tamarin serum was layered onto 10⁵ cells in multiwell-6-wells 1 day after plating at multiplicity of infection of 0.75 G.E./cell. After 6 hours adsorption, the virus-containing serum was removed, the cells extensively washed and incubated in the serum-free DMEM. Cells from individual wells were lyzed with Trizol at different times and GBV-B RNA quantified by Taqman.

As an alternative method, 10⁶ cells were mixed with undiluted virus-containing serum (10⁶ G.E.) and subjected to electroporation. The cells were plated at a density of 10⁵/well and treated as described for experiments of RNA electroporation. Duplicate wells were run in parallel, with and without interferon. Cells from individual wells were lyzed with Trizol at different times and GBV-B RNA quantified by Taqman. The amount of G.E. detectable immediately after electroporation and wash was 10³ G.E., it was undetectable at 24 and 48 hours, at day 3 it was 10⁴ G.E.; at day 6 it was 5×10³ (possibly inhibition due to cell density); in corresponding wells treated with interferon the RNA was undetectable. At day 6 IFN-treated cells were discarded, non-treated cells were split 1:6 (and duplicates were run +/− interferon), at day 7 the RNA raised again to 1 G.E., decreased at almost undetectable levels at day 10 (inhibition may have been due to cell density). At day 10 the non-treated cells were split again 1:6. RNA was quantified at day 13 and 16 giving a figure corresponding to 4×10³ G.E. and 1.2×10⁴ G.E. respectively. The last point (16 days) value corresponds to about 0.1 G.E. per cell. Corresponding points obtained with cells cultivated in the presence of IFN gave background signals.

### Example 10

### Production of Additional Replicon Enhanced Cells

Human hepatoma cell lines, HepG2 and Hep3B, were transfected with neo-RepB RNA. Permissive cell lines were observed with Hep3B (obtained from the ATCC), but not with HepG2. Eleven Hep3B permissive cell lines were further characterized. Hep3B was obtained from the ATCC.

The 11 cell lines show a gradient of permissiveness to GBV-B with respect to parental Hep3B. The most enhanced is the cell line designated “RepB/Hep3B-9”. A cell line with an intermediate phenotype is “RepB/Hep3B-11”. Mutations were identified for the replicons from RepB/Hep3B-9 and RepB/Hep3B-11 (Table 2).

TABLE 2

| Cell line | Nucleotide | Gene | Amino acid |
|---|---|---|---|
| RepB/3B-9 | “A” insert in “A” | EMCV IRES | — |
| | stretch 1876-1880 | | |
| | A2780G | NS3 (hel) | Ala296 silent |
| RepB/3B-11 | T1827G | EMCV IRES | |
| | T2375A | NS3(pro) | Ser161, silent |

### Example 11

### Production of Replicon Enhanced Cells Supporting A Genomic Replicon

A GBV-B selectable full length replicon (neo-FL-D) was constructed by inserting the genes encoding core-E1-E2-NS2 between EMCV IRES and the GBV-B NS3 in neo-RepD. RNA transcribed from this clone was electroporated into cB76/Huh7 cells. Three cell lines bearing the full-length replicon were isolated, the presence and replication of neo-FL-D was confirmed by qPCR, and normal PCR.

Mutations from three different cell lines supporting neo-FL-D are shown in Table 3.

TABLE 3

| Cell Line | Mut. Gene | Nucleotide | Amino acid |
|---|---|---|---|
| FL-D/Huh7-1.1 | NS3 | T5222C | Ala156, silent |
| FL-D/Huh7-2.1 | NS5B | A10417G | Glu554Gly |
| FL-D/Huh7-3.1 | Neo | G1050T | Arg177Leu |
| | Core | G2197C | Gly88Ala |

All three cell lines were cured with IFN. This treatment gave rise to second generation cured cells. The second generation cells were re-transfected with neo-RepB, neo-FL-D, bla-FL-D (beta-lactamase encoding sequence replacing neo in neo-FL-D), and HCV replicons (Con1 replicon w.t. sequence and NS5A A227T corresponding mutant). Con 1 is described by Lohmann et al., Science 285:110-113, 1999.

Neo-FL-D and bla-FL-D efficiently replicated only in the second-generation-cured cFL/Huh7 cell lines (the best was that produced curing FL-D/Huh7-1.1 and FL-D/Huh7-2.1). The second-generation-cured cells were not improved compared to parental cB76.1/Huh7 for replication of the subgenomic neo-RepB replicon. The cells were enhanced for w.t Con1 HCV replicon and, at a higher extent, for NS5A A227T mutant HCV replicon compared to cB76.1/Huh7 (the best was that produced curing FL-D/Huh7-3.1).

Other embodiments are within the following claims. While several embodiments have been shown and described, various modifications may be made without departing from the spirit and scope of the present invention.

1. A GBV-B replicon comprising the following regions:
a GBV-B 5′ UTR substantially similar to bases 1-445 of SEQ ID NO 1;
a selection or reporter sequence functionally coupled to said GBV-B 5′ UTR;
an internal ribosome entry site;
a NS3-NS5B sequence substantially similar to bases 1938-7709 of SEQ ID NO: 1 functionally coupled to said internal ribosome entry site and an AUG translation initiation codon; and
a GBV-B 3′ UTR substantially similar to bases 7710-8069 of SEQ ID NO: 1,
wherein said replicon is capable of replication in a cell.

2. The GBV-B replicon of claim 1, further comprising a GBV-B structural region, wherein said GBV-B structural region is functionally coupled to said GBV-B 5′UTR.

3. The GBV-B replicon of claim 2, wherein
said GBV-B structural region comprises a sequence substantially similar to a sequence selected from the group consisting of: bases 446-511 of SEQ ID NO: 1, bases 446-487 of SEQ ID NO: 1, bases of 446-469 of SEQ ID NO: 1, the RNA version of bases 446-2641 of SEQ ID NO: 2, and the RNA version of bases 446-3265 of SEQ ID NO: 2.

4. The GBV-B replicon of claim 3, wherein said replicon consists of:
said GBV-B 5′UTR;
said GBV-B structural region;
said selection or reporter sequence;
said internal ribosome entry site;
said NS3-NS5B sequence; and
said GBV-B 3′ UTR.

5. The GBV-B replicon of claim 4, wherein
said internal ribosome entry site has the sequence of bases 1324-1934 of SEQ ID NO 1;
said GBV-B structural region consisting of a sequence selected from the group consisting of: bases 446-511 of SEQ ID NO: 1, bases 446-487 of SEQ ID NO: 1, bases of 446-469 of SEQ ID NO 1, the RNA version of bases 446-2642 of SEQ ID NO: 2 and the RNA version of bases 446-3265 of SEQ ID NO: 2;
said NS3-NS5B region is Met-NS3-NS5B region consisting of bases 1935-7709 of SEQ ID NO: 1; and
said GBV-B 3′ UTR is bases 7710-8069 of SEQ ID NO: 1.

6. The GBV-B replicon of claim 5, wherein said GBV-B structural region consists either of the RNA version of bases 446-2642 of SEQ ID NO: 2 or the RNA version of bases 446-3265 of SEQ ID NO: 2.

7. The GBV-B replicon of claim 1, wherein said replicon consists of SEQ ID NO: 1.

8. The GBV-B replicon of claim 2, wherein
said GBV-B structural region comprises a sequence substantially similar to a sequence selected from the group consisting of: bases 446-511 of SEQ ID NO: 1, bases 446-487 of SEQ ID NO: 1, bases of 446-469 of SEQ ID NO: 1, and the RNA version of bases 446-2641 of SEQ ID NO: 2.

9. The GBV-B replicon of claim 3, wherein said replicon consists of:
said GBV-B 5′ UTR;
said selection or reporter sequence;
said internal ribosome entry site;
said GBV-B structural region;
a NS2-NS5B region comprising a NS2 region substantially similar to the RNA version of bases 2642-3265 of SEQ ID NO: 2 joined to the 5′ end of said NS3-NS5B region; and
said GBV-B 3′ UTR.

10. The GBV-B replicon of claim 9, wherein
said internal ribosome entry site has the sequence of 1324-1934 of SEQ ID NO 1;
said GBV-B structural region comprises a sequence selected from the group consisting of: bases 446-511 of SEQ ID NO 1, bases 446-487 of SEQ ID NO 1, bases of 446-469 of SEQ ID NO 1, and the RNA version of bases 446-2641 of SEQ ID NO: 2;
said NS2-NS5B is a Met-NS2-NS5B region consisting of said 5′ AUG translation initiation codon, said NS2 region, and said NS3-NS5B region, wherein said NS2 region consists of the RNA version of bases 2642-3265 of SEQ ID NO: 2 and said NS3-NS5B consists of bases 1938-7709 of SEQ ID NO: 1; and
said GBV-B 3′ UTR is bases 7710-8069 of SEQ ID NO: 1.

11. The GBV-B replicon of claim 10, wherein said replicon produces an infectious virion.

12. An expression vector comprising a promoter transcriptionally coupled to a nucleotide sequence coding the GBV-B replicon of claim 1.

13. A GBV-B replicon made by a process comprising the steps of transfecting a cell with the replicon of claim 1 and isolating said replicon.

14. The GBV-B replicon of claim 13, wherein said cell is either a Huh7 cell, a Hep3B cell, is derived from a Huh7 cell, or is derived from a Hep3B cell.

15. A method of making a second GBV-B replicon from a first GBV-B replicon comprising the steps of:
a) transfecting a cell with said first replicon, wherein said first replicon is the replicon of claim 1;
b) isolating a replicon from said transfected cell;
c) determining the nucleotide sequence of said replicon from said transfected cell; and
d) producing said second replicon, wherein said second replicon contains the first replicon sequence with one or more alterations corresponding to said replicon from said transfected cell.

16. The method of claim 15, wherein said cell is either a Huh7 cell, a Hep3B cell, is derived from a Huh7 cell, or is derived from a Hep3B cell.

17. A method of measuring the ability of a compound to affect GBV-B replicon activity comprising the steps of:
a) providing said compound to a cell containing the GBV-B replicon of claim 1; and
b) measuring the ability of said compound to affect one or more replicon activities as a measure of the effect on GBV-B replicon activity.

18. The method of claim 17, wherein said cell is a human hepatoma cell.

19. The method of claim 18, wherein said said cell is either a Huh7 cell, a Hep3B cell, is derived from a Huh7 cell, or is derived from a Hep3B cell.

20. A GBV-B replicon enhanced cell, wherein said cell has an maintenance and activity efficiency of at least 25% when transfected with a GBV-B replicon of SEQ ID NO: 1 by the Electroporation Method.

21-23. (canceled)

24. A method of making a GBV-B replicon enhanced cell comprising the steps of:
a) introducing and maintaining the GBV-B replicon of claim 1 in a cell; and
b) curing said cell of said GBV-B replicon to produce said replicon enhanced cell.

25-26. (canceled)

27. A method of making a GBV-B replicon enhanced cell containing a functional GBV-B replicon comprising the steps of:
a) introducing and maintaining a first GBV-B replicon in a cell, wherein said first replicon is the replicon of claim 1;
b) curing said cell of said first replicon to produce a cured cell; and
c) introducing and maintaining a second GBV-B replicon into said cured cell, wherein said second GBV-B replicon may be the same or different than said first GBV-B replicon.

28-47. (canceled)
